(19) United States
(12) Patent Application Publication
(10) Pub. No.: US 2023/0285517 A1
(43) Pub. Date: Sep. 14, 2023

(54) COMPOSITIONS COMPRISING ENZYMES AND PROBIOTICS, AND METHODS FOR PREVENTING OR TREATING MACULAR DEGENERATION

(71) Applicant: GenoFocus, Inc., Yuseong-gu (KR)

(72) Inventors: Jae Gu Pan, Sejong (KR); Eui Joong Kim, Daejeon (KR); Jeong Hyun Kim, Seongnam-Si (KR); Do Young Yum, Daejeon (KR)

(21) Appl. No.: 17/923,296

(22) PCT Filed: May 5, 2021
(86) PCT No.: PCT/IB2021/000303

§ 371 Date: Nov. 4, 2022

Related U.S. Application Data
(60) Provisional application No. 63/020,241, filed on May 5, 2020.

(51) Int. Cl. A61K 38/44; A61K 35/742; A61K 45/06; A61P 27/02; C12N 9/02

(52) U.S. Cl. CPC A61K 38/446; A61K 35/742; A61K 45/06; A61P 27/02; C12N 9/0089; C12Y 115/01001

(57) ABSTRACT

The present invention relates, in part, to methods of preventing or treating macular degeneration in a subject by co-administering a superoxide dismutase enzyme and probiotic Bacillus sp. spores, especially a Bacillus amyloliquefaciens GF423 or GF424 mutant strain. The present invention also provides pharmaceutical and/or food compositions comprising a superoxide dismutase enzyme and probiotic Bacillus sp. spores.

### RELATED APPLICATION

This application claims the benefit of priority to U.S. Provisional Application No. 63/020,241, filed on May 5, 2020, the contents of which is incorporated herein by reference in its entirety.

### TECHNICAL FIELD

The present invention provides methods of preventing or treating macular degeneration by coadministering superoxide dismutase (SOD) enzyme and probiotic Bacillus sp. spores. Also provided herein are pharmaceutical or food compositions comprising SOD enzyme and probiotic Bacillus sp. spores for preventing or treating macular degeneration.

### BACKGROUND

Age-related macular degeneration (“AMD”) refers to the chronic, progressive degenerative pathology of the macula, which results in loss of central vision. Macular degeneration is a major cause of vision loss and irreversible central vision loss in adults over 50 years of age. More than 25 million people around the world suffer from AMD, and the number of these people continues to grow rapidly due to the rapid growth of the elderly population. In addition, excessive use of electronic devices such as smartphones and laptops also causes the early onset and increased prevalence of macular degeneration in people today.

The most important causes of age-related macular degeneration (AMD) are age-related atrophy and a decline in the function of retinal pigment epithelium (RPE), which plays a critical role in maintaining the homeostasis and physiological function of the retina that plays a key role in visual function. In addition, the age-related abnormal changes in Bruch’s membrane and degeneration of choroidal capillaries are also thought to contribute to the etiology of AMD. Bruch’s membrane functions as the basement membrane of the RPE, while choroidal capillaries are located on the outermost side of the neural retina and supply nutrients and oxygen to photoreceptor cells in which photoconversion occurs.

The age-related macular degeneration is largely classified into two categories: dry macular degeneration characterized by the degeneration and functional decline of RPE, Bruch’s membrane, and choroidal capillaries; and wet macular degeneration which involves choroidal neovascularization (CNV) in addition to the symptoms of dry macular degeneration.

Wet macular degeneration occurs in 5 to 10% of patients with dry macular degeneration and can lead to acute blindness within months if left untreated. This is in contrast to dry macular degeneration in which vision deterioration progresses over a period of a few years or about ten to twenty years.

In wet macular degeneration, there is a widespread decrease in oxygen partial pressure and nutrients across the subretinal space and the sub-retinal pigment epithelial (RPE) space, leading to ischemia in tissues accompanied by an inflammatory response.

In addition, the complement system, which plays an important role in oxidative stress and immune response, acts such that choroidal neovascularization (CNV) characteristically occurs in the subretinal space and the sub-retinal pigment epithelial (RPE) space, causing serous leakage and hemorrhage.

It is known that vascular endothelial cells, RPE cells, and inflammatory cells such as monocytes and macrophages are involved in the development of choroidal neovascularization.

Potential treatment for macular degeneration includes anti-angiogenic agents such as a decorin peptide (PCT Publication No. WO 2005/116066; incorporated by reference) or a conjugate thereof (U.S. Pat. Application No. 2009/0246133 A1; incorporated by reference). However, such agents have not shown to be effective against choroidal neovascularization or age-related macular degeneration.

The clinical standard of care for wet AMD is an antibody therapy against vascular endothelial growth factor (VEGF). While it has been effective in reducing blindness in many patients, the anti-VEGF antibody or a fragment thereof (e.g., aflibercept) has not been able to completely inhibit the formation and growth of choroidal neovascularization, in part due to its action being limited to the epithelial cells on the surface of neovascular vessels. Moreover, the antibody has not been effective in preventing the eventual loss of functional photoreceptor cells in the central foveal of the retina, resulting from disruption of the underlying RPE tissue. Furthermore, the anti-VEGF antibody is administered by intravitreal injection, causing fear and side effects in patients.

Accordingly, there is a great need for oral compositions and methods for effectively treating macular degeneration without the intravitreal injection.

### SUMMARY

The present invention is based, at least in part, on the discovery that oral co-administration of a superoxide dismutase (SOD) enzyme in combination with probiotic Bacillus sp. spores is more effective than SOD alone in preventing and treating macular degeneration (e.g., wet macular degeneration).

SOD is an antioxidant enzyme that removes reactive oxygen species, a major cause of AMD. While attempts have been made in the past to administer orally the SOD enzyme to treat ocular diseases, it has not conferred a protective effect against light-induced oxidative stress (Sicard et al. (2006) Investigative Ophthalmology & Visual Science 47:2089). Similarly, the oral administration of GliSODin® comprising mellon extracts enriched with SOD failed to protect against the onset of neovascular AMD in human (Hera et al. (2009) Investigative Ophthalmology & Visual Science 50:258). Moreover, GliSODin® further comprises gliadin (a wheat protein), a known risk factor for celiac disease, thereby limiting the treatable patient population.

SOD alone was surprisingly effective in preventing and treating wet macular degeneration. The compositions and methods provided herein further comprising probiotic Bacillus sp. spores are even more effective in preventing and treating wet macular degeneration. In some embodiments, by formulating with shellac, the SOD enzyme is protected from the gastric acid upon being administered orally. Thus, the compositions and methods of the present disclosure can deliver orally an effective amount of active SOD, thereby eliminating the need for the intravitreal injection and simplifying the therapeutic modality of AMD treatment. In addition, in some embodiments, the SOD enzyme of the present disclosure is sourced from generally regarded as safe (GRAS) bacteria with proven safety.

Continuing to emphasize oral availability and GRAS bacteria sourced probiotics, Bacillus sp. spores are resistant to gastric protease and low pH. Also, Bacillus sp. spores are GRAS probiotics approved in several countries. It was conceived that combining SOD with probiotic Bacillus sp. spores would enhance the treatment efficacy of SOD and also in reducing the amount of SOD enzymes needed. Combination treatment of SOD with probiotic spores was found to be surprisingly even more effective than SOD alone, not only in improving the treatment efficacy but also in improving consistency of therapeutic efficacy among the treated individual subject animals. More importantly, the compositions and methods provided herein are highly effective in inhibiting CNV and restoring retinal function. Thus, these methods and oral compositions comprising SOD enzyme and probiotic Bacillus sp. spores are highly effective in preventing or treating wet macular degeneration.

### BRIEF DESCRIPTION OF FIGURES

FIG. 1 shows a schematic diagram of a mouse study to evaluate the in vivo effect of a pharmaceutical composition comprising SOD enzyme and probiotic Bacillus sp. spores.

FIG. 2 depicts fundus fluorescein angiography images (upper panel), showing the changes in CNV lesions after administration of test substance(s) (spore derived from Bacillus amyloliquefaciens strain GF424 (GF203); 10U or 20U of GF-101 (the composition comprising SOD); combination of GF101 and GF203; 20 µg of aflibercept (AF; a positive control); phosphate buffered saline (PBS; a negative control)). The bottom panel shows a graph showing CTF values.

FIG. 3 shows retinal tomography images obtained by an optical coherence tomography performed on laser-induced CNV mice administered with test substance(s). The images show changes in the size of CNV lesions after the administration of test substance(s).

FIG. 4 shows the size of CNV lesions calculated from retinal tomography images obtained by an optical coherence tomography, which was performed on laser-induced CNV mice administered with a test substance(s).

FIG. 5 shows the results of electroretinography on mouse CNV models that were irradiated with a laser and then subsequently treated with a test substance(s).

FIG. 6 shows the changes in electroretinography b-wave amplitudes of mouse CNV models that were irradiated with a laser and then subsequently administered with a test substance(s).

FIG. 7 shows the histological analysis of mouse CNV models that were irradiated with a laser and then administered with a test substance(s). The tissues were stained with Haemotoxylin and Eosin (H & E) for observation.

FIG. 8 shows the results of a TUNEL assay demonstrating a decreased number of dead cells in retinas of the mouse CNV models irradiated with a laser and then treated with a test substance(s).

FIG. 9 shows the results of immunofluorescence staining performed to examine changes in the expression of VEGF after laser irradiation and administration of various test substances.

FIG. 10 shows the results of immunofluorescence staining performed to examine changes in the expression of STAT3 after laser irradiation and administration of various test substances.

FIG. 11 shows the results of Western hybridization performed to examine changes in the expression of HIF-1α and NRF2 after laser irradiation and administration of various test substances. (A) Western blotting and (B) quantitative comparison of the level of HIF-1α and NRF2 in retina.

### DETAILED DESCRIPTION

The present invention relates, in part, to compositions and methods for preventing and treating macular disorder (e.g., AMD, wet AMD). It is discovered herein that an oral composition comprising SOD enzyme and probiotic Bacillus sp. spores is more effective than SOD alone in inhibiting choroidal neovascularization (CNV) associated with wet AMD.

In certain aspects, provided herein is a method of treating or preventing macular degeneration, comprising administering to a subject in need thereof a superoxide dismutase (SOD) enzyme and probiotic Bacillus sp. spores (e.g., Bacillus coagulans, Bacillus subtilis, Bacillus indicus, Bacillus clausii, Bacillus licheniformis, Bacillus amyloliquefaciens).

In some embodiments, the SOD enzyme is an isolated enzyme and/or is a recombinant enzyme. In some embodiments, the SOD enzyme binds manganese. In some embodiments, the SOD enzyme comprises: (a) the amino acid sequence with at least or about 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more identity to the sequence set forth in SEQ ID NO: 1; (b) the amino acid sequence set forth in SEQ ID NO: 1, wherein the amino acid residue Asn74 and/or Asn137 is deleted or substituted; (c) the amino acid sequence set forth in SEQ ID NO: 1, wherein the amino acid residue Asn74 and/or Asn137 is substituted with Asp74 and/or Asp137; or (d) the amino acid sequence set forth in SEQ ID NO: 1. In some embodiments, the SOD enzyme is coated with shellac.

In some embodiments, the SOD enzyme and/or the Bacillus sp. spores are administered orally, intravenously, intraocularly, or intramuscularly. In preferred embodiments, the SOD enzyme and/or the Bacillus sp. spores are administered orally.

In some embodiments, the SOD enzyme is from a microorganism, preferably a bacterium, preferably a bacterium generally regarded as safe (GRAS) for use as food and drug, more preferably a Bacillus species bacterium. In some embodiments, the SOD enzyme is from Bacillus amyloliquefaciens GF423 strain (KCTC 13222BP) or from GF424 strain (KCTC 13227BP).

In some embodiments, the probiotic Bacillus sp. spores are generally regarded as safe (GRAS) for use as food and approved drugs, preferably the spores of a Bacillus amyloliquefaciens GF423 strain or GF424 mutant strain.

In certain embodiments, the method (i) decreases choroidal neovascularization (CNV); (ii) decreases cell death in the retina; (iii) decreases inflammation in the retina; (iv) decreases hypoxia in the retina; (v) decreases the expression of vascular endothelial growth factor (VEGF) in the retina; and/or (vi) increases retinal function.

In some embodiments, the macular degeneration is an age-related macular degeneration (AMD), preferably wherein the AMD is a wet AMD or a neovascular AMD.

In some embodiments, the subject is a mammal, preferably wherein the mammal is a human, a dog, a cat, a mouse, or a rat. In preferred embodiments, the subject is a human.

In some embodiments, the SOD enzyme and the probiotic Bacillus sp. spores are administered to the subject sequentially.

In other embodiments, the SOD enzyme and the probiotic Bacillus sp. spores are administered to the subject simultaneously. In some embodiments, the subject is administered with a composition comprising the SOD enzyme and the probiotic Bacillus sp. spores.

In some embodiments, the SOD enzyme and/or the Bacillus sp. spores are in a pharmaceutical composition or a nutraceutical composition.

In some embodiments, the method further comprises administering to the subject at least one additional agent that treats macular degeneration. In some embodiments, the at least one additional agent is ranibizumab or aflibercept.

In certain aspects, also provided herein is a method of decreasing or inhibiting choroidal neovascularization (CNV), comprising contacting a retina with a SOD enzyme and probiotic Bacillus sp. spores (e.g., Bacillus coagulans, Bacillus subtilis, Bacillus indicus, Bacillus clausii, Bacillus licheniformis, Bacillus amyloliquefaciens). In some embodiments, the method is performed in vivo, ex vivo, or in vitro.

In some embodiments, the SOD enzyme is an isolated enzyme and/or is a recombinant enzyme. In some embodiments, the SOD enzyme binds manganese. In some embodiments, the SOD enzyme comprises: (a) the amino acid sequence with at least or about 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more identity to the sequence set forth in SEQ ID NO: 1; (b) the amino acid sequence set forth in SEQ ID NO: 1, wherein the amino acid residue Asn74 and/or Asn137 is deleted or substituted; (c) the amino acid sequence set forth in SEQ ID NO: 1, wherein the amino acid residue Asn74 and/or Asn137 is substituted with Asp74 and/or Asp137; or (d) the amino acid sequence set forth in SEQ ID NO: 1. In some embodiments, the SOD enzyme is coated with shellac.

In some embodiments, the SOD enzyme is from a microorganism, preferably a bacterium, preferably a bacterium generally regarded as safe (GRAS) for use as food and drug, more preferably a Bacillus species bacterium. In some embodiments, the SOD enzyme is from Bacillus amyloliquefaciens GF423 strain (KCTC 13222BP) or from GF424 strain (KCTC 13227BP).

In some embodiments, the probiotic Bacillus sp. spores are generally regarded as safe (GRAS) for use as food and approved drugs, preferably the spores of a Bacillus amyloliquefaciens GF423 strain or GF424 mutant strain.

In certain embodiments, the method (i) decreases cell death in the retina; (ii) decreases inflammation in the retina; (iii) decreases hypoxia in the retina; (iv) decreases the expression of vascular endothelial growth factor (VEGF) in the retina; and/or (v) increases retinal function.

In some embodiments, the retina is of a subject afflicted with a macular degeneration. In some embodiments, the retina is of a subject afflicted with an age-related macular degeneration (AMD), preferably wherein the AMD is a wet AMD or a neovascular AMD.

In some embodiments, the retina is of a mammal, preferably wherein the mammal is a human, a dog, a cat, a mouse, or a rat. In preferred embodiments, the mammal is a human.

In some embodiments, the SOD enzyme and the probiotic Bacillus sp. spores contact the retina sequentially.

In other embodiments, the SOD enzyme and the probiotic Bacillus sp. spores contact the retina simultaneously. In some embodiments, the retina is contacted with a composition comprising the SOD enzyme and the probiotic Bacillus sp. spores.

In some embodiments, the SOD enzyme and/or the Bacillus sp. spores are in a pharmaceutical composition or a nutraceutical composition. In some embodiments, the SOD enzyme and/or the probiotic Bacillus sp. spores are in a pharmaceutical composition.

In some embodiments, the method further comprises contacting the retina with at least one additional agent that decreases or inhibits CNV. In some embodiments, the at least one additional agent is ranibizumab or aflibercept.

In certain aspects, provided herein is a pharmaceutical composition comprising a superoxide dismutase (SOD) enzyme and probiotic Bacillus sp. spores (e.g., Bacillus coagulans, Bacillus subtilis, Bacillus indicus, Bacillus clausii, Bacillus licheniformis, Bacillus amyloliquefaciens).

In some embodiments, the SOD enzyme is an isolated or purified enzyme. In some embodiments, the SOD enzyme is a recombinant enzyme. In some embodiments, the SOD enzyme binds manganese. In some embodiments, the SOD enzyme comprises: (a) the amino acid sequence with at least or about 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more identity to the sequence set forth in SEQ ID NO: 1; (b) the amino acid sequence set forth in SEQ ID NO: 1, wherein the amino acid residue Asn74 and/or Asn137 is deleted or substituted; (c) the amino acid sequence set forth in SEQ ID NO: 1, wherein the amino acid residue Asn74 and/or Asn137 is substituted with Asp74 and/or Asp137; or (d) the amino acid sequence set forth in SEQ ID NO: 1. In some embodiments, the SOD enzyme is coated with shellac.

In some embodiments, the composition is an oral composition.

In some embodiments, the SOD enzyme is from a microorganism, preferably a bacterium, preferably a bacterium generally regarded as safe (GRAS) for use as food and drug, more preferably a Bacillus species bacterium. In some embodiments, the SOD enzyme is from Bacillus amyloliquefaciens GF423 strain (KCTC 13222BP) or from GF424 strain (KCTC 13227BP).

In some embodiments, the probiotic Bacillus sp. spores are generally regarded as safe (GRAS) for use as food and approved drugs, preferably the spores of a Bacillus amyloliquefaciens GF423 strain or GF424 mutant strain.

In some embodiments, the composition further comprises at least one additional agent that decreases or inhibits CNV; or at least one additional agent that treats macular degeneration. In some embodiments, the macular degeneration is an age-related macular degeneration (AMD), preferably wherein the AMD is a wet AMD or a neovascular AMD. In some embodiments, the at least one additional agent is ranibizumab or aflibercept.

In certain embodiments, the composition (i) decreases choroidal neovascularization (CNV); (ii) decreases cell death in the retina; (iii) decreases inflammation in the retina; (iv) decreases hypoxia in the retina; (v) decreases the expression of vascular endothelial growth factor (VEGF) in the retina; and/or (vi) increases retinal function.

In certain aspects, further provided herein is a medical or nutraceutical food comprising a superoxide dismutase (SOD) enzyme and probiotic Bacillus sp. spores (e.g., Bacillus coagulans, Bacillus subtilis, Bacillus indicus, Bacillus clausii, Bacillus licheniformis, Bacillus amyloliquefaciens).

In some embodiments, the SOD enzyme is an isolated or purified enzyme. In some embodiments, the SOD enzyme is a recombinant enzyme. In some embodiments, the SOD enzyme binds manganese. In some embodiments, the SOD enzyme comprises: (a) the amino acid sequence with at least or about 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more identity to the sequence set forth in SEQ ID NO: 1; (b) the amino acid sequence set forth in SEQ ID NO: 1, wherein the amino acid residue Asn74 and/or Asn137 is deleted or substituted; (c) the amino acid sequence set forth in SEQ ID NO: 1, wherein the amino acid residue Asn74 and/or Asn137 is substituted with Asp74 and/or Asp137; or (d) the amino acid sequence set forth in SEQ ID NO: 1. In some embodiments, the SOD enzyme is coated with shellac.

In some embodiments, the SOD enzyme is from a microorganism, preferably a bacterium, preferably a bacterium generally regarded as safe (GRAS) for use as food and drug, more preferably a Bacillus species bacterium. In some embodiments, the SOD enzyme is from Bacillus amyloliquefaciens GF423 strain (KCTC 13222BP) or from GF424 strain (KCTC 13227BP).

In some embodiments, the probiotic Bacillus sp. spores are generally regarded as safe (GRAS) for use as food and approved drugs, preferably the spores of a Bacillus amyloliquefaciens GF423 strain or GF424 mutant strain.

In some embodiments, the medical or nutraceutical food further comprises at least one additional agent that decreases or inhibits CNV; or at least one additional agent that treats macular degeneration. In some embodiments, the macular degeneration is an age-related macular degeneration (AMD), preferably wherein the AMD is a wet AMD or a neovascular AMD. In some embodiments, the at least one additional agent is ranibizumab or aflibercept.

In certain embodiments, the medical or nutraceutical food (i) decreases choroidal neovascularization (CNV); (ii) decreases cell death in the retina; (iii) decreases inflammation in the retina; (iv) decreases hypoxia in the retina; (v) decreases the expression of vascular endothelial growth factor (VEGF) in the retina; and/or (vi) increases retinal function.

In certain aspects, provided herein is a pharmaceutical composition comprising probiotic Bacillus sp. spores (e.g., Bacillus coagulans, Bacillus subtilis, Bacillus indicus, Bacillus clausii, Bacillus licheniformis, Bacillus amyloliquefaciens).

In some embodiments, the probiotic Bacillus sp. spores are generally regarded as safe (GRAS) for use as food and approved drugs. In some embodiments, the probiotic Bacillus sp. spores are the spores of a Bacillus amyloliquefaciens GF423 strain or GF424 mutant strain.

In some embodiments, the composition further comprises at least one additional agent that decreases or inhibits CNV; or at least one additional agent that treats macular degeneration. In some embodiments, the macular degeneration is an age-related macular degeneration (AMD), preferably wherein the AMD is a wet AMD or a neovascular AMD. In some embodiments, the at least one additional agent is ranibizumab or aflibercept.

In certain aspects, also provided herein is a medical or nutraceutical food comprising probiotic Bacillus sp. spores (e.g., Bacillus coagulans, Bacillus subtilis, Bacillus indicus, Bacillus clausii, Bacillus licheniformis, Bacillus amyloliquefaciens).

In some embodiments, the probiotic Bacillus sp. spores are generally regarded as safe (GRAS) for use as food and approved drugs. In some embodiments, the probiotic Bacillus sp. spores are the spores of a Bacillus amyloliquefaciens GF423 strain or GF424 mutant strain.

In some embodiments, the medical or nutraceutical food further comprises at least one additional agent that decreases or inhibits CNV; or at least one additional agent that treats macular degeneration. In some embodiments, the macular degeneration is an age-related macular degeneration (AMD), preferably wherein the AMD is a wet AMD or a neovascular AMD. In some embodiments, the at least one additional agent is ranibizumab or aflibercept.

In certain aspects, provided herein is a kit comprising any one or combination of pharmaceutical compositions described herein, and/or any one or combination of the medical or nutraceutical food described herein.

### Definitions

The articles “a” and “an” are used herein to refer to one or to more than one (i.e. to at least one) of the grammatical object of the article. By way of example, “an element” means one element or more than one element.

The term “administering” is intended to include routes of administration which allow therapy to perform its intended function. Examples of routes of administration include oral administration, sublingual administration, and intravitreal administration. As used herein, the term “age-related macular degeneration” or “AMD” includes early, intermediate, and advanced AMD, and also includes both dry macular degeneration, geographic atrophy, and wet macular degeneration, also known as neovascular or exudative AMD.

The terms “conjoint therapy” and “combination therapy,” as used herein, refer to the administration of two or more therapeutic substances. The different agents comprising the combination therapy may be administered concomitant with, prior to, or following the administration of one or more therapeutic agents.

As used herein, the terms “prevent,” “preventing,” and “prevention” are art-recognized, and when used in relation to a medical condition such as a loss of vision, or a disease such as macular degeneration, is well understood in the art, and include administration of a composition which reduces the frequency of, or delays the onset of, symptoms of a medical condition (e.g., blurry vision or a loss of vision) in a subject relative to a subject which does not receive the composition.

The term “subject” or “patient” refers to any healthy or diseased animal, mammal or human, or any animal, mammal or human. In some embodiments, the subject is afflicted with macular degeneration (e.g., neovascular macular degeneration). In various embodiments of the methods of the present invention, the subject has not undergone treatment. In other embodiments, the subject has undergone treatment.

As used herein, the term “therapeutically effective amount” of the composition or agent refers to an amount of an agent which provides the desired effect, such as reducing, preventing or slowing the progression of physical changes associated with macular degeneration in the eye, or reducing, preventing or slowing the progression of symptoms (e.g., accumulation of drusen, abnormal blood vessel growth in the eye, abnormal fluid in the eye, blood and protein leakage, etc.) resulting from them. The exact amount of agent required may vary from subject to subject depending on the species, age and general condition of the subject, mode of administration, and the like. However, an appropriate “effective amount” in any individual case may be determined by one of ordinary skill in the art using routine experimentation.

The term “treating” includes prophylactic and/or therapeutic treatments. The term “prophylactic or therapeutic” treatment is art-recognized and includes administration to the host of one or more of the subject compositions. If it is administered prior to clinical manifestation of the unwanted condition (e.g., disease or other unwanted state of the host animal), then the treatment is prophylactic (i.e., it protects the host against developing the unwanted condition); whereas, if it is administered after manifestation of the unwanted condition, the treatment is therapeutic (i.e., it is intended to diminish, ameliorate, or stabilize the existing unwanted condition or side effects thereof).

### Macular Degeneration

The pathogenesis of AMD is still incompletely understood due to various factors. Aging of retinal pigment epithelial layer (RPE) cells and Bruch’s membrane, impaired blood flow in the vascular membrane of the eye, retinal exposure to ultraviolet light and blue light, and genetic predisposition are believed to play an important role in the development of AMD.

The loss of RPE cells, which appears in the early stage of AMD, is mainly due to oxidative stress, which results from weakening of the antioxidant cell defense system or increased concentration of reactive oxygen species, and thus effective removal of reactive oxygen species may be essential for prevention and treatment of AMD.

1 to 5% of the total oxygen consumption in the body is converted into reactive oxygen species (ROS), which are the major source of oxidative stress. An imbalance between routine production and detoxification of reactive oxygen species (“ROS”) such as peroxides and free radicals can result in oxidative damage to cellular structures and machinery. The human retina consumes a large amount of oxygen, and in particular, retinal pigment epithelial cells produce a large amount of reactive oxygen species because these cells phagocytose the visual cell outer segment. In addition, intracellular reactive oxygen species are also produced through the mitochondrial electron transport system. Oxidative stress-induced retinal pigment epithelial cells undergo induced apoptosis or show changes such as mitochondrial DNA damage, increased vascular endothelial growth factor (VEGF), decreased antioxidant enzymes, and increased inflammatory responses.

### Superoxide Dismutase (SOD)

Superoxide dismutase (SOD) is an enzyme that alternately catalyzes the dismutation of the superoxide (O₂-) radical into either ordinary molecular oxygen (O₂) or hydrogen peroxide (H₂O₂). Thus, SODs play a key role in decreasing oxidative stress by removing reactive oxygen species. SODs are widely distributed in prokaryotic and eukaryotic cells and have been classified into four families based on their different types of metal centers [copper/zinc, nickel, manganese, and iron]. Manganese-containing SODs [Mn-SODs] are widely present in many bacteria, chloroplasts, mitochondria, and cytosol of eukaryotic cells. The SOD enzyme derived from B. amyloliquefaciens GF423 strain (KCTC 13222BP) is a Mn-SOD and has the amino acid sequence of SEQ ID NO: 1. The SOD enzyme derived from B. amyloliquefaciens GF424 strain (KCTC 13227BP) is a Mn-SOD and also has the amino acid sequence of SEQ ID NO: 1.

### Isolation/Purification of SOD

An “isolated” or “purified” SOD or biologically active portion thereof is substantially free of cellular material or other contaminating proteins from the cell or tissue source from which the enzyme is derived. The language “substantially free of cellular material” includes preparations of a polypeptide, in which the protein is separated from cellular components of the cells from which it is isolated or recombinantly produced. In some embodiments, the language “substantially free of cellular material” includes preparations of protein, having less than about 30% (by dry weight) of non-desired protein, more preferably less than about 20% of non-desired protein, still more preferably less than about 10% of non-desired protein, and most preferably less than about 5% non-desired protein.

SOD can be isolated or purified from various sources, including natural or recombinant hosts. For example, SOD having an activity of preventing or treating macular degeneration disease can be extracted from the culture supernatant of the B. amyloliquefaciens GF423 strain or the B. amyloliquefaciens GF424 strain. First, a culture can be obtained by culturing the B. amyloliquefaciens GF423 strain or the B. amyloliquefaciens GF424 strain in various types of media. In some embodiments, a complex medium (pH 6.0 to 7.0) is used to grow the bacteria at 25 to 42° C. for 1 to 4 days. Other suitable media for culturing the B. amyloliquefaciens GF423 strain or the B. amyloliquefaciens GF424 strain include LB (Luria-Bertani) medium, ISP (International Streptomyces Project) medium, NA (nutrient agar) medium, BHI (brain heart infusion agar) medium, SDA (sabouraud dextrose agar) medium, PDA (potato dextrose agar) medium, NB (nutrient broth) medium, and the like. In preferred embodiments, LB medium, ISP medium, BHI medium, SDA medium, or NB medium may be used.

SOD may also be sourced from other natural or recombinant hosts using the information provided in databases such as PubMed or BRENDA (world wide web at brenda-enzymes.org).

The SOD is preferably purified by the following purification method but is not limited thereto. A culture obtained by culturing the B. amyloliquefaciens GF423 strain or the B. amyloliquefaciens GF424 strain is centrifuged to collect the culture supernatant. The supernatant fraction is pretreated by solid-phase extraction and then isolated and purified by chromatography. Various modes of chromatography may be used to purify SOD. In preferred embodiments, a hydrophobic interaction chromatography is used.

### Bacillus Sp. Spores

In certain aspects, provided herein are spores of Bacillus Sp. and compositions (e.g., pharmaceutical composition, nutraceutical composition) comprising the said spores of Bacillus Sp. Further provided herein are use of such spores and/or compositions in the treatment of a subject and/or decreasing or inhibiting neovascularization (CNV). In preferred embodiments, the spores of Bacillus Sp. are used conjointly with the SOD enzyme of the present disclosure.

Spore-forming bacilli produce a large number of secretory proteins, enzymes, antimicrobial compounds, vitamins, and carotenoids (Elshaghabee et al. (2017) Frontiers in Microbiology 8:1490). For this reason, spore-forming bacilli have been used in food chain (e.g., as probiotics). However, these bacteria or their spores have not been implicated in the methods (e.g., for treatment of the diseases described herein) of the present disclosure. In some embodiments, exemplary probiotic Bacillus Sp. include Bacillus coagulans, Bacillus subtilis, Bacillus indicus, Bacillus clausii, Bacillus licheniformis, and Bacillus amyloliquefaciens. In preferred embodiments, the probiotic Bacillus Sp. is Bacillus amyloliquefaciens (e.g., GF423 or GF424).

### Pharmaceutical Composition

The composition of the present invention may further comprise a conventional pharmaceutically acceptable carrier or excipient. In addition, the SOD enzyme derived from the B. amyloliquefaciens GF423 or G424 strain may be formulated with various additives, such as a binder, a coating agent and the like, which are pharmaceutically commonly used.

The pharmaceutical composition containing the SOD according to the present invention may contain a pharmaceutically acceptable carrier. For oral administration, the pharmaceutically acceptable carrier may include a binder, a lubricant, a disintegrant, an excipient, a solubilizer, a dispersing agent, a stabilizer, a suspending agent, a coloring agent, a flavoring agent, and the like. For topical administration, the pharmaceutically acceptable carrier may include a base, an excipient, a lubricant, a preservative, and the like. The pharmaceutical composition of the present invention may be formulated into a variety of dosage forms in combination with the aforementioned pharmaceutically acceptable carriers. For example, for oral administration, the pharmaceutical composition may be formulated in solid or liquid dosage forms such as tablets, troches, capsules, elixirs, suspensions, syrups, wafers, or the like. In addition, the pharmaceutical composition may be formulated into solutions, suspensions, tablets, capsules, sustained-release preparations, or the like.

Meanwhile, examples of the carrier, excipient, and diluent suitable for formulation may include lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methylhydroxy benzoate, propylhydroxy benzoate, talc, magnesium stearate, mineral oil, or the like. In addition, the pharmaceutical composition may further contain a filler, an anti-agglutinating agent, a lubricating agent, a wetting agent, a flavoring agent, an emulsifying agent, a preservative, or the like.

In the method of the present invention, the SOD enzyme may be coated with shellac. When the SOD is administered orally, a problem may arise in that the activity of the SOD is reduced rapidly in the gastrointestinal tract, leading to a decrease in the bioavailability and efficiency thereof. This problem is further exacerbated by the difficulty of delivering the SOD to the particular cell location where the SOD is most effective. Thus, in the method of the present invention, the SOD enzyme may be coated in a solution. Specifically, a purified solution and a shellac-containing solution are mixed with each other, and then freeze-dried. This freeze-dried sample may be powdered and stored at about 4° C. until use. Examples of coatings suitable for use in the present invention include shellac, ethyl cellulose, hydroxypropyl methylcellulose, hydroxypropyl methylcellulose phthalate, zein, Eudragit, and combinations thereof.

### Dose

The dose of the pharmaceutical composition of the present invention, which contains the SOD produced from the B. amyloliquefaciens GF423 or G424 strain, may be suitably determined in consideration of the purpose of treatment or prevention, the type of patient to be prevented or treated, the patient’s condition, weight, age or sex, etc. For example, the composition of the present invention may contain, as an active ingredient, the SOD produced by the B. amyloliquefaciens GF423 or GF424 strain and probiotic Bacillus sp. spores in a therapeutically effective amount or at a nutritionally effective concentration. Preferably, the composition may contain the SOD in an amount of 2 to 3000 U/mg, based on the total weight of the composition and varying amount of probiotic Bacillus sp. spores.

### A Medical or Nutraceutical Food

Still another aspect of the present invention provides a food, particularly a nutraceutical food, or medical foods, for preventing, ameliorating or treating macular degeneration and a degenerative decline in eye function, the food containing a SOD derived from the B. amyloliquefaciens GF423 or GF424 strain. The SOD from the B. amyloliquefaciens GF423 has the amino acid sequence of SEQ ID NO: 1. The SOD from the B. amyloliquefaciens GF424 also has the amino acid sequence of SEQ ID NO: 1.

As used herein, the term “nutraceutical food” or “medical food” means a food prepared with such a raw material or a component that is likely to be beneficial function for human body, which is defined by Ministry of Food and Drug Safety as the food to maintain or improve health by maintaining the normal function or by activating the physiological function of the human body, but not always limited thereto and does not exclude any conventional health food in its meaning.

The nutraceutical or medical food of the present invention may be prepared and processed in the form of tablets, capsules, powders, granules, liquids, pills, or the like, for the purpose of preventing or ameliorating macular degeneration. Conventional additives include, for example, chemical synthetic additives, such as ketones, glycine, calcium citrate, nicotinic acid, cinnamic acid, and the like; natural additives, such as persimmon color, licorice extract, crystalline cellulose, kaoline pigment, guar gum, and the like; and mixed formulations, such as L-sodium glutamate formulations, alkali additives for noodles, preservative formulations, tar color formulations, and the like. For example, a nutraceutical food in the form of a tablet may be prepared by granulating a mixture of the active ingredient SOD of the present invention with an excipient, a binder, a disintegrating agent and other additives by a conventional method, and then adding a lubricant, or the like thereto, followed by compression molding, or directly compression-molding the mixture. In addition, the nutraceutical food in the form of a tablet may contain a corrigent, or the like, if necessary.

Among nutraceutical foods in the form of a capsule, a hard capsule formulation may be prepared by filling a hard capsule with a mixture of the active ingredient SOD or bacterial strain powder of the present invention with an additive, such as an excipient. A soft capsule formulation may be prepared by filling a mixture of the SOD or the strain powder with an additive, such as an excipient, into a capsule such as a gelatin capsule. The soft capsule formulation may, if necessary, contain a plasticizer, such as glycerin or sorbitol, a coloring agent, a preservative, or the like.

A nutraceutical food in the form of a pill may be prepared by molding a mixture of the active ingredient SOD of the present invention with an excipient, a binder, a disintegrant, and the like by a known method. The pill formulation may, if necessary, be coated with white sugar or other coating agent or may also be surface-coated with a substance such as starch or talc.

### Conjoint or Combination Therapy

The combination therapy can be sequential therapy, wherein the subj ect is treated first with the SOD enzyme and then the probiotic Bacillus sp. spores or vice versa. These can be administered independently by the same route or by two different routes of administration depending on the dosage forms employed.

The SOD enzyme and the probiotic Bacillus sp. spores can be administered simultaneously as part of a single composition.

The SOD enzyme and the probiotic Bacillus sp. spores can be administered simultaneously as separate compositions. These can be administered independently by the same route or by two different routes of administration depending on the dosage forms employed.

The compositions provided herein contain a combination (e.g., SOD enzyme and probiotic Bacillus sp. spores) of active agents that are useful in treating macular degeneration.

The combination of active agents described herein can be combined with one or more other pharmacologically active compounds known in the art according to the methods and compositions provided herein. It is believed that certain combinations work synergistically in the treatment of macular degeneration (e.g., wet AMD) or in the inhibition of CNV.

The additional active agents can be large molecules (e.g., proteins) or small molecules (e.g., synthetic inorganic, organometallic, or organic molecules). In some embodiments, at least one additional therapy that may be combined with the SOD and probiotic Bacillus sp. spores is an agent that can treat macular degeneration or an agent that can decrease or inhibit CNV. In some embodiments, the agent is approved by the U. S. Food and Drug Administration. In some such embodiments, the agent is afilbercept, an inhibitor of VEGF. In other such embodiments, the agent is ranibizumab, another inhibitor of VEGF.

In some embodiments, the compositions provided herein are used as a primary treatment. In other embodiments, the compositions are used as adjuvant therapy.

In some such embodiments, the compositions provided herein may be administered to a subject before, concurrently, or after the administration of the one or more other pharmacologically active compounds.

### Sequence Identity / Homology

Function-conservative variants are those in which a given amino acid residue in a protein or enzyme has been changed without altering the overall conformation and function of the polypeptide, including, but not limited to, replacement of an amino acid with one having similar properties (such as, for example, polarity, hydrogen bonding potential, acidic, basic, hydrophobic, aromatic, and the like). Amino acids other than those indicated as conserved may differ in a protein so that the percent protein or amino acid sequence similarity between any two proteins of similar function may vary and may be, for example, from 70% to 99% as determined according to an alignment scheme such as by the Cluster Method, wherein similarity is based on the MEGALIGN algorithm. A function-conservative variant also includes a polypeptide which has at least 60% amino acid identity as determined by BLAST or FASTA algorithms, preferably at least 75%, more preferably at least 85%, still preferably at least 90%, and even more preferably at least 95%, and which has the same or substantially similar properties or functions as the native or parent protein to which it is compared.

The percent identity between two sequences is a function of the number of identical positions shared by the sequences (i.e., % identity= # of identical positions/total # of positions x 100), taking into account the number of gaps, and the length of each gap, which need to be introduced for optimal alignment of the two sequences. The comparison of sequences and determination of percent identity between two sequences can be accomplished using a mathematical algorithm, as described in the nonlimiting examples below.

The percent identity between two nucleotide sequences can be determined using the GAP program in the GCG software package (available on the world wide web at the GCG company website), using a NWSgapdna. CMP matrix and a gap weight of 40, 50, 60, 70, or 80 and a length weight of 1, 2, 3, 4, 5, or 6. The percent identity between two nucleotide or amino acid sequences can also be determined using the algorithm of E. Meyers and W. Miller (CABIOS, 4:11 17 (1989)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. (48):444 453 (1970)) algorithm which has been incorporated into the GAP program in the GCG software package (available on the world wide web at the GCG company website), using either a Blosum 62 matrix or a PAM250 matrix, and a gap weight of 16, 14, 12, 10, 8, 6, or 4 and a length weight of 1, 2, 3, 4, 5, or 6.

The nucleic acid and protein sequences of the present invention can further be used as a “query sequence” to perform a search against public databases to, for example, identify related sequences. Such searches can be performed using the NBLAST and XBLAST programs (version 2.0) of Altschul, et al. (1990) J. Mol. Biol. 215:403 10. BLAST nucleotide searches can be performed with the NBLAST program, score=100, wordlength=12 to obtain nucleotide sequences homologous to the nucleic acid molecules of the present invention. BLAST protein searches can be performed with the XBLAST program, score=50, wordlength=3 to obtain amino acid sequences homologous to the protein molecules of the present invention. To obtain gapped alignments for comparison purposes, Gapped BLAST can be utilized as described in Altschul et al., (1997) Nucleic Acids Res. 25(17):3389 3402. When utilizing BLAST and Gapped BLAST programs, the default parameters of the respective programs (e.g., XBLAST and NBLAST) can be used (available on the world wide web at the NCBI website).

### Sequences

As used herein, coding region refers to regions of a nucleotide sequence comprising codons which are translated into amino acid residues, whereas noncoding region refers to regions of a nucleotide sequence that are not translated into amino acids (e.g., 5′ and 3′ untranslated regions).

Complement [to] or complementary refers to the broad concept of sequence complementarity between regions of two nucleic acid strands or between two regions of the same nucleic acid strand. It is known that an adenine residue of a first nucleic acid region is capable of forming specific hydrogen bonds (base pairing) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. In some embodiments, the first region comprises a first portion and the second region comprises a second portion, whereby, when the first and second portions are arranged in an antiparallel fashion, at least or about 50%, and preferably at least or about 75%, at least or about 90%, or at least or about 95% of the nucleotide residues of the first portion are capable of base pairing with nucleotide residues in the second portion. In other embodiments, all nucleotide residues of the first portion are capable of base pairing with nucleotide residues in the second portion.

A nucleic acid is operably linked when it is placed into a functional relationship with another nucleic acid sequence. For instance, a promoter or enhancer is operably linked to a coding sequence if it affects the transcription of the sequence. With respect to transcription regulatory sequences, operably linked means that the DNA sequences being linked are contiguous and, where necessary to join two protein coding regions, contiguous and in reading frame. For switch sequences, operably linked indicates that the sequences are capable of effecting switch recombination.

There is a known and definite correspondence between the amino acid sequence of a particular protein and the nucleotide sequences that can code for the protein, as defined by the genetic code (shown below). Likewise, there is a known and definite correspondence between the nucleotide sequence of a particular nucleic acid and the amino acid sequence encoded by that nucleic acid, as defined by the genetic code.

GENETIC CODE

| | |
|---|---|
| Alanine (Ala, A) | GCA, GCC, GCG, GCT |
| Arginine (Arg, R) | AGA, ACG, CGA, CGC, CGG, CGT |
| Asparagine (Asn, N) | AAC, AAT |
| Aspartic acid (Asp, D) | GAC, GAT |
| Cysteine (Cys, C) | TGC, TGT |
| Glutamic acid (Glu, E) | GAA, GAG |
| Glutamine (Gln, Q) | CAA, CAG |
| Glycine (Gly, G) | GGA, GGC, GGG, GGT |
| Histidine (His, H) | CAC, CAT |
| Isoleucine (Ile, I) | ATA, ATC, ATT |
| Leucine (Leu, L) | CTA, CTC, CTG, CTT, TTA, TTG |
| Lysine (Lys, K) | AAA, AAG |
| Methionine (Met, M) | ATG |
| Phenylalanine (Phe, F) | TTC, TTT |
| Proline (Pro, P) | CCA, CCC, CCG, CCT |
| Serine (Ser, S) | AGC, AGT, TCA, TCC, TCG, TCT |
| Threonine (Thr, T) | ACA, ACC, ACG, ACT |
| Tryptophan (Trp, W) | TGG |
| Tyrosine (Tyr, Y) | TAC, TAT |
| Valine (Val, V) | GTA, GTC, GTG, GTT |
| Termination signal (end) | TAA, TAG, TGA |

An important and well-known feature of the genetic code is its redundancy, whereby, for most of the amino acids used to make proteins, more than one coding nucleotide triplet may be employed (illustrated above). Therefore, a number of different nucleotide sequences may code for a given amino acid sequence. Such nucleotide sequences are considered functionally equivalent since they result in the production of the same amino acid sequence in all organisms (although certain organisms may translate some sequences more efficiently than they do others). Moreover, occasionally, a methylated variant of a purine or pyrimidine may be found in a given nucleotide sequence. Such methylations do not affect the coding relationship between the trinucleotide codon and the corresponding amino acid.

In making the changes in the amino sequences of polypeptide, the hydropathic index of amino acids may be considered. The importance of the hydropathic amino acid index in conferring interactive biologic function on a protein is generally understood in the art. It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of their hydrophobicity and charge characteristics these are: isoleucine (+4.5); valine (+4.2); leucine (+3.8); phenylalanine (+2.8); cysteine/cystine (+2.5); methionine (+1.9); alanine (+1.8); glycine (-0.4); threonine (-0.7); serine (-0.8); tryptophane (-0.9); tyrosine (-1.3); proline (-1.6); histidine (-3.2); glutamate (-3.5); glutamine (-3.5); aspartate (<RTI 3.5); asparagine (-3.5); lysine (-3.9); and arginine (-4.5).

It is known in the art that certain amino acids may be substituted by other amino acids having a similar hydropathic index or score and still result in a protein with similar biological activity, i.e. still obtain a biological functionally equivalent protein.

As outlined above, amino acid substitutions are generally therefore based on the relative similarity of the amino acid side-chain substituents, for example, their hydrophobicity, hydrophilicity, charge, size, and the like. Exemplary substitutions which take various amino acids of the foregoing characteristics into consideration are well-known to those of skill in the art and include: arginine and lysine; glutamate and aspartate; serine and threonine; glutamine and asparagine; and valine, leucine and isoleucine.

In view of the foregoing, the nucleotide sequence of a DNA or RNA can be used to derive the polypeptide amino acid sequence, using the genetic code to translate the DNA or RNA into an amino acid sequence. Likewise, for polypeptide amino acid sequence, corresponding nucleotide sequences that can encode the polypeptide can be deduced from the genetic code (which, because of its redundancy, will produce multiple nucleic acid sequences for any given amino acid sequence). Thus, description and/or disclosure herein of a nucleotide sequence which encodes a polypeptide should be considered to also include description and/or disclosure of the amino acid sequence encoded by the nucleotide sequence. Similarly, description and/or disclosure of a polypeptide amino acid sequence herein should be considered to also include description and/or disclosure of all possible nucleotide sequences that can encode the amino acid sequence.

### Kits

The present invention also encompasses kits. For example, the kit can comprise an engineered or natural polypeptide of the present disclosure (e.g., SOD enzyme), Bacillus sp. spores, a pharmaceutical composition as described herein, medical or nutraceutical food as described herein, a combination therapy including e.g., at least one additional agent that treats macular degeneration or decreases or inhibits CNV, for example, ranibizumab or aflibercept, or any combination thereof, packaged in a suitable container and can further comprise instructions for using such reagents. The kit may also contain other components, such as administration tools packaged in a separate container.

### EXAMPLES

### Example 1. Strain Isolation and Identification

### 1) 16S rRNA Analysis

From Bacillus polyfermenticus purchased from Bi-Nex Co., Ltd., a strain was isolated (“the Strain”), and the Strain was identified and characterized as described below.

To characterize the Strain, a morphological and biochemical examination was performed. The morphological examination of the Gram stained bacteria indicated that the Strain was a Gram-positive bacillus. In addition, observation under a phase contrast microscope showed that the Strain formed endospores.

To determine the identity of the Strain, 16s rRNA sequencing was performed as follows. The genome of the Strain was purified (Sambrook, J. et al.: “Molecular Cloning. A Laboratory Manual, 3rd ed.,” 2001, Cold Spring Harbor Press), and sequenced using Illumina HiSeq PE100. Nine copies of the 16S rRNA gene (SEQ ID NOs: 2 to 10) were found. Among the 16S rRNA genes, BPJGP_r00130 (SEQ ID NO: 7) and BPJGP_r00160 (SEQ ID NO: 8) showed the same nucleotide sequence, but other 16S rRNA genes showed different nucleotide sequences. Thus, the Strain had eight 16S rRNA genes with distinct nucleotide sequences.

With 9 copies of the 16S rRNA gene, analysis for the genus identification was performed using the following database and softwares: The Ribosomal Database Project’s Classifier (Wang, Q. et al., Appl Environ Microbiol., 73:5261-5267 (2007)), Living Tree Project’s Aligner (Pruesse, E. et al., Bioinformatics, 28:1823-1829 (2012)), and EzTaxon database’s Identity (Kim, O. S. et al., Int J Syst Evol Microbiol., 62:716721 (2012)). The Strain was identified to be a member of the genus Bacillus according to all the software listed above, with a confidence interval of 95% or more.

Species level identification of the isolated strain was performed using the EzTaxon database’s Identity (Kim, O. S. et al., Int J Syst Evol Microbiol., 62:716721 (2012)). Although there is currently no international standard for the identity threshold of 16S rRNA for species level identification, 99% is the highest value of the most widely accepted thresholds (Yarza, P. et al., Nature Rev. Microbiol., 12: 635645 (2014)). Accordingly, the 99% threshold was used as a search standard. In addition, since the Strain had eight distinct 16S rRNA genes, a search was performed for each of the 16S rRNA genes. Among the found reference strains, the commonly found reference strains were selected. The search identified 80 different reference strains belonging to different species. This result is consistent with previous studies indicating that species belonging to the genus Bacillus cannot be distinguished using only the homology of 16S rRNA genes (Janda J. M. & Abbott S. L., J Clin Microbiol., 45:2761-2764 (2007); Maughan H. & Van der Auwera G., Infect Genet Evol., 11:789-797 (2011)).

Thus, in order to determine the identity of the Strain, a genome-based classification was performed. The homology between the Strain and the 80 strains identified above was analyzed using the in silico DNA-DNA Hybridization (DDH; Auch A. F. et al., Stand Genomic Sci., 28:117-234(2010)), and the reference strains showing a homology of greater than 70% were selected. Two reference strains were found in the analysis (see Table 1 below), and their ANI (the average nucleotide identity) and AAI (the average amino acid identity) at the genomic level with respect to the Strain were verified (Rodriguez-R L.M. & Konstantinidis K. T., Peer J Preprints 4:e1900v1 (2016)).

Table 1 below shows the analysis of the 16S rRNA gene, DDH, ANI and AAI of three strains, which showed the highest homology with the Strain in the DDH analysis.

TABLE 1

| | Bacillus amyloliquefaciens plantarum | Bacillus amyloliquefaciens amyloliquefaciens | Bacillus subtilis spizizenii | Criteria for species classification |
|---|---|---|---|---|
| Reference strain | FZB42 | DSM7 | NRRL B-23049 | |
| 16S rRNA | 99.67 to 99.73% | 99.46 to 99.66% | 99.18 to 99.39% | 98.65% |
| DDH | 92.10% | 78.60% | 32.70% | 70% |
| ANI | 98.65% | 93.59% | 80.04% | 95% |
| AAI | 98.79% | 95.09% | 79.88% | 95% |

Genome-based comparison described above identified the Strain as a microorganism belonging to B. amyloliquefaciens. The Strain was named Bacillus amyloliquefaciens GF423 and deposited with the Korean Collection for Type Cultures (KCTC), a patent strain depository authority, on Mar. 6, 2017, under accession number KCTC 13222BP.

### Example 2. Production of Bacillus Amyloliquefaciens GF424 Mutant Strain

To improve the expression of the sodA gene, the Bacillus amyloliquefaciens GF423 strain was mutated by UV irradiation. From the UV-mutant library, a Bacillus amyloliquefaciens GF424 mutant strain having 4.5-fold higher SOD activity than that of the wild-type strain was selected. It was confirmed by sequencing that the sodA gene of Bacillus amyloliquefaciens GF424 was the same as that of the wild-type strain. The Bacillus amyloliquefaciens GF424 mutant strain was cultured in tryptic soy medium at 37° C. (BD). PCR was performed with Takara’s Advantage 2 Polymerase by a standard method.

The mutant strain obtained as described above was named Bacillus amyloliquefaciens GF424 and deposited with the Korean Collection for Type Cultures (KCTC), a patent strain depository authority, on Mar. 23, 2017 under accession number KCTC 13227BP.

### Example 3. Isolation/Purification of Superoxide Dismutase (SOD) From Bacillus Amyloliquefaciens GF423 or GF424

### 3.1 Culturing of Bacillus Amyloliquefaciens GF423 Strain

For culturing of the Bacillus amyloliquefaciens GF423 strain, a single colony formed in LB agar medium (Luria-Bertani (LB) agar; 10 g/L tryptophan, 5 g/L yeast extract, 10 g/L NaCl, 15 g/L agar) was inoculated into 30 mL of LB medium and cultured at 37° C. for 12 hours. The seed culture was inoculated again into 3L of LB medium containing 1 mM manganese sulfate (MnSO₄) and was cultured at 37° C. for 20 hours. Then, a portion of the culture was used for the separation of SOD. The remaining portion was diluted at 10¹¹ CFU/mL in phosphate buffered saline (PBS, 10 mM sodium phosphate, 130 mM sodium chloride, pH 7.4) and sonicated, and then the supernatant was collected by centrifugation, filtered through a filter having a pore size of 0.45 µm, freeze-dried, and then stored at -20° C. until use in an in vivo experiment.

The Bacillus amyloliquefaciens GF424 strain can also be cultured using the method described above.

### 3.2 Isolation and Purification of Superoxide Dismutase

The culture of the B. amyloliquefaciens GF423 strain was centrifuged at 3,578xg at 4° C. for 20 minutes and the supernatant was collected and concentrated 10-fold by ultrafiltration (MWCO 10,000). Ammonium sulfate was added to 300 mL of the concentrated supernatant to a saturation degree of 60% with stirring at 4° C., followed by stirring for 30 minutes. Then, the supernatant was collected by centrifugation at 3,578xg for 30 minutes, and loaded onto a HiPrep™ Phenyl HP 16/10 column equilibrated with 50 mM potassium phosphate (pH 7.5) containing 2 M ammonium sulfate. Next, elution was performed using 50 mM potassium phosphate (pH 7.5) containing 2 M to 0.1 M ammonium sulfate. The SOD-containing fraction was collected, concentrated by UF (MWCO 10,000), and desalted by dialysis with 50 mM potassium phosphate (pH 7.5). The activity of the SOD was analyzed using a SOD assay kit (Cayman Chemical, Michigan, USA). One unit of SOD activity is defined as the amount of enzyme that inhibits superoxide radicals by 50%. The activity of the purified SOD enzyme was 2231.12±269 U/mg, and the molecular weight of the SDS was about 22,000 Dalton.

The SOD derived from the B. amyloliquefaciens GF423 was coated with the natural coating agent shellac. Shellac was dissolved in 50 mM potassium phosphate (pH 7.0) buffer, mixed with a purified solution of the SOD, and freeze-dried. The freeze-dried sample was in a powder form and stored at 4° C. The SOD derived from the B. amyloliquefaciens GF423 strain was designated as GF-101.

The SOD enzyme from the Bacillus amyloliquefaciens GF424 strain can also be produced, isolated, and purified using the method described above.

### Example 4. A Variant of SOD, GF-103

Deamidation of some populations of Asn74 and Asn137 residues in the purified GF-101 was found by peptide mapping with trypsin digest and amino acid sequencing analysis: 21.8% for Asn74 and 11.3% for Asn137. Table 2A summarizes the deamidation sites and the peptides harboring the sites with the amino acid sequence of GF-101. The two Asn residues were substituted for Asp to improve the homogeneity of the purified enzyme. The variant SOD was designated as GF-103. Peptide mapping of GF-103 showed that there was no unexpected peptide. Subsequent amino acid sequencing of the peptides (Table 2B) confirmed the results of peptide mapping. The substitutions of Asn to Asp did not affect enzyme activity and/or stability.

TABLE 2A

| | | | | | | | | | | |
|---|---|---|---|---|---|---|---|---|---|---|
| 1 | AYKLP | ELPYA | YDALE | PHIDK | ETMTI | HHTKH | HNTYV | TNLNK | AIEGS | ALAEK |
| | | | | | ▼▼ Deamidation | | | | | |
| 51 | SVDEL | VADLN | AVPED | IRTAV | RNNGG | GHANH | SLFWT | LLSPN | GGGEP | TGELA |
| | | | | | | T8 | | ▼▼ Deamidation | | |
| 101 | EEIKS | TFGSF | DQFKE | KFAAA | AAGRF | GSGWA | WLVVN | NGKLE | ITSTP | NQDSP |
| | | | | | | | T12 | | | |
| 151 | LSEGK | TPVLG | LDVWE | HAYYL | NYQNR | RPDYI | SAFWN | VVNWD | EVARL | YSEAK |

TABLE 2B

| Theoretical Mass (I) | | | | | GF103_190811 | | |
|---|---|---|---|---|---|---|---|
| Frag# | Res# | Modification | m/z | Charge | m/z | RT | Sequencing |
| T1 | 1-3 | | 381.21 | 1 | 381.21 | 1.8 | AYK |
| T2 | 4-20 | | 662.01 | 3 | 662.00 | 41.0 | LPHPYAYDALEPHIDK |
| T3 | 21-23 | | 543.27 | 2 | 549.27 | 68 | ETMTIHHTK |
| T2-3 | 4-29 | Miss-cleavage | 513.31 | 5 | 613.31 | 68.3 | LPELPYAYDALEPHIDKETMTIHHIK |
| T2-3(F) | 4-23 | Fragment Miss-cleavage | 782.38 | 3 | 782.38 | 43.1 | LPELPYAYDALEPHIDKETM |
| T1-3 | 1-29 | | 856.94 | 4 | 856.93 | 38.6 | AYKLPELPYAYDALEPHIDKETMTIHHTK |
| T4 | 30-40 | | 447.56 | 3 | 447.56 | 12.7 | HHNYYVTNLNK |
| T5 | 41-50 | | 494.77 | 2 | 494.77 | 14.5 | AIEGSALAEK |
| T6 | 51-67 | | 927.98 | 2 | 927.97 | 44.4 | SVDELVADLNAVPEDIR |
| T7 | 68-71 | | 446.27 | 1 | 446.27 | 1.9 | TAVR |
| T8 | 72-104 | | 851.91 | 4 | 851.90 | 47.7 | NDGGGHANHSLFWTLLSPNGGGEFTGELAEEIK |
| T9 | 105-114 | | 582.27 | 2 | 582.27 | 61.0 | STFGSFOFK |
| T10 | 115-116 | | 276.16 | 1 | N.D | N.D | EK |
| T3-10 | 105-116 | Miss-cleavage | 474.23 | 3 | 474.23 | 27.6 | STFGSFDGFKEK |
| T11 | 117-124 | | 367.70 | 2 | 367.70 | 10.7 | FAAAAAGR |
| T12 | 125-138 | | 768.39 | 2 | 768.36 | 46.2 | FGSGWAWLVVNDGK |
| T13 | 139-155 | | 908.45 | 2 | 908.45 | 24.2 | LBTSTPNQDSPLSEGK |
| T12-13 | 125-155 | Miss-cleavage | 1111.55 | 3 | 1111.54 | 50.6 | FGSGWAWLVVNDGKLEITSTPNQDSPLSEGK |
| T14 | 156-175 | | 817.74 | 3 | 817.74 | 47.3 | TPVLGDVWEHAYYLNYGNR |
| T15 | 176-194 | | 779.72 | 3 | 779.71 | 53.3 | RPDWSAFWNVVNWDEVAR |
| T16 | 195-200 | | 355.69 | 2 | 355.69 | 6.4 | LYSEAK |

### Example 5. Preparation of Spore Bacillus Amyloliquefaciens GF424 Mutant Strain

### Media Composition

Medium used was SYP or DSM. SYP media contains 1.5% soy tone, 0.5% yeast extract, 0.5% K₂HPO₄, 0.1% MnSO₄, 0.1% MgSO₄, 10 mM FeSO₄, 0.04% (NH₄)₂SO₄, 0.04% (NH₄)₂PO₄, 0.1% CaCl₂, and 2% glucose. DSM media contains 8 g/L bacto nutrient broth, 1 g/L KCl, 0.25 g/L MgSO₄, 0.16415 g/L Ca(NO₃)₂, 0.9521 mg/L MnCl₂, and 0.152 mg FeSO₄. MnSO₄, MgSO₄ FeSO₄, (NH₄)₂SO₄, (NH₄)₂PO₄, and CaCl₂ were dissolved in ddH₂O and added prior to use.

### Sporulation Induction

Single colony of Bacillus amyloliquefaciens strain GF424 was inoculated into 1 mL of LB in 14 mL tube and incubated at 37° C., 200 rpm for 12 h. 1 mL of the culture was transferred to 50 mL of LB medium in 500 mL flask and incubated at 37° C., 200 rpm for 12 h. Then, 20 mL of cultured medium was transferred to 1 L of SYP or DSM in 2.5 L baffled flasks. Inoculated cultures were incubated at 37° C., 200 rpm for 24 h up to 120 h.

### Spore Washing

After cultivation, lysozyme (0.5 g/L) was added to culture broth, and incubated at 37° C., 200 rpm for 1h for removal of remaining vegetative cells. Crude spore was harvested by centrifugation at 6000 rpm for 10 min. The crude spore was further purified as follows: washing 2 times with water, washing with 0.02% SDS, washing 2 times with water and then suspended in PBS solution. The spore suspension was stored at -20° C. The number of spore was determined by counting the colonies after spreading diluted spore solution on LB agar plates.

### Example 6. Evaluation of Choroidal Neovascular Inhibitory Effect of Superoxide Dismutase (SOD) Derived from Bacillus Amyloliquefaciens GF423

### 6.1. Experimental Animals and Construction of Choroidal Neovascular (CNV) Models

Animal experiments were performed in accordance with the Animal Use and Care Protocol of the Institutional Animal Care and Use Committee (IACUC). C57BL/6 mice were purchased from Koatech Co., Ltd. and acclimated for 14 days. Then, the mice were raised for 17 days at an average temperature of 19 to 25° C., a humidity of 40 to 60% and an average illuminance of 150 to 300 lux with a 12-hr light/12-hr dark cycle. The mice were given feed and water ad libitum daily.

7-week-old C57BL/6 mice were anesthetized with a mixture of ketamine hydrochloride (40 mg/kg) and xylazine hydrochloride (10 mg/kg), and then the Bruch’s membrane of the mouse eye was irradiated with a diode green laser (532 nm, 150 mW, 0.1 sec, 50 µM), thereby inducing choroidal neovascularization.

### 6.2. Administration of Test Substances

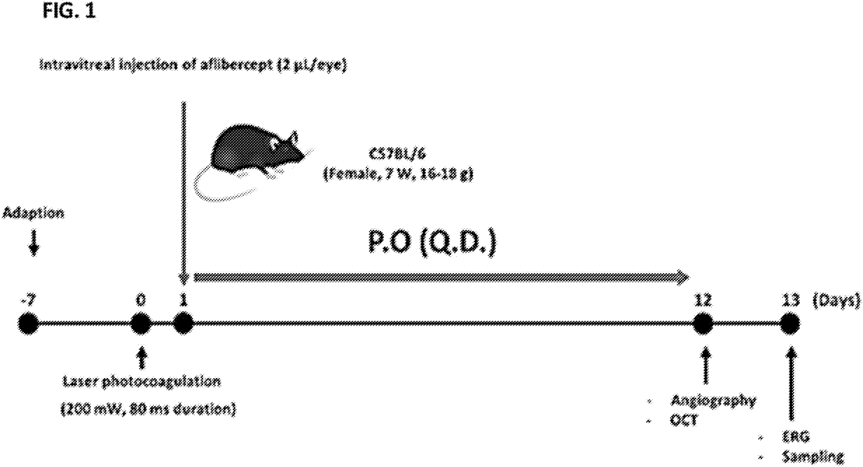

Experimental animals were grouped as described below, irradiated with a laser (day 0), and administered test substance(s) from day 1 (FIG. 1).

Aflibercept is a product approved by the US Food and Drug Administration (FDA) for use as an agent for treating age-related macular degeneration. To a negative control group and a CNV-induced group (test group II), PBS as a placebo was administered as described below. GF-101 is SOD derived from the B. a. GF423 strain. GF-203 is spore prepared from B. a. GF424 strain
Test group I (NC): naive control group.
Test group II: group administered with PBS after CNV induction. 100 µL of PBS was orally administered from Day 1 to Day 12.
Test group III (PC): group administered with aflibercept. 20 µg of aflibercept was injected intravitreally into both eyes.
Test group IV: group administered with GF-203 after CNV induction. 100 µL of GF203 dissolved in PBS (10⁷ cfu) was orally administered from Day 1 to Day 12.
Test group V: group administered with GF-101 after CNV induction. 100 µL of GF101 dissolved in PBS (10 U) was orally administered from Day 1 to Day 12.
Test group VI: group administered with GF-101 after CNV induction. 100 µL of GF101 dissolved in PBS (20 U) was orally administered from Day 1 to Day 12.
Test group VII: group administered with GF-101 (10 U) + GF203 after CNV induction. 100 µL of GF203 dissolved in PBS (10⁷ cfu) and 100 µL of GF101 dissolved in PBS (10 U) were orally administered from Day 1 to Day 12.
GF-203: Aliquots of 1 mL were stored in a test substance freezer (-20° C.) of the test institute, and then taken out once a day immediately before administration and 100 µl was administered to each animal.
GF-101 (20 U): Aliquots of GF-101 were stored a test substance freezer (4° C.) of the test institute, and then taken out once a day immediately before administration. A solution having a concentration of 200 U/mL was prepared by mixing 26.6 mg of GF-101 with 2 mL of PBS, and 100 µL of the solution was administered to each animal.
GF-101 (10 U): A solution having a concentration of 200 U/mL was prepared by diluting GF-101 (200 U/mL) two-fold, and 100 µL of the solution was administered to each animal.
GF-203+GF-101 (10 U): 1 mL of a GF-203 preparation was mixed with 1 mL of GF-101 (100 U/mL), and 200 µL of the mixture was administered to each animal.

### 6.3. Animal Test

### Fundus Fluorescein Angiography (FFA)

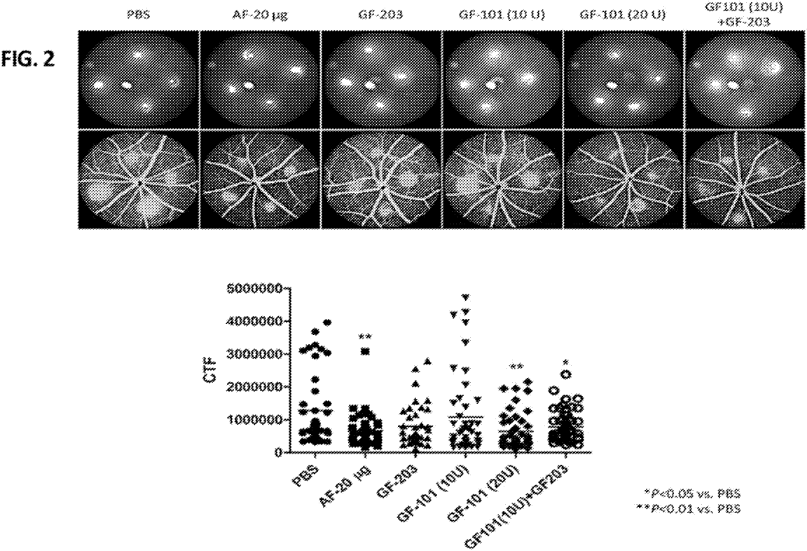

Fluorescein leakage from choroidal neovascularization was measured using fundus fluorescein angiography (FFA). Fundus fluorescent angiography was performed using a micron IV imaging system. 2% fluorescein was injected intraperitoneally into the mice of each test group under anesthesia, and after waiting for 3 to 5 minutes, the pupils were dilated, fundus fluorescein angiography (FFA) imaging was performed, the background was corrected, and the CTF values were calculated. As shown in FIG. 2, it was observed that choroidal neovascularization (CNV) lesions were formed 12 days after laser irradiation.

After administration of the pharmaceutical composition of the present invention, the area of CNV in the eye of the mice, measured by fundus fluorescence angiography, was decreased compared to the CNV area before the start of treatment. The decreased retinal thickness is a decreased central retinal subfield thickness (CST), a decreased center point thickness (CPT), or a decreased central foveal thickness (CFT).

The CTF value of the group administered intraocularly with the positive control aflibercept (AF) (test group III) was 673,595 ± 486,147, compared to that of the PBS-administered group (test group II) (1,279,587 ± 1,094,827), and the CNV area was decreased by 52.6% compared to that of the PBS-administered group. The GF-203-administered group (test group IV) (799,849 ± 635,299), the GF-101 (10 U)-administered group (test group V) (1,124,635 ± 1,249,267) and the GF-101 (20 U)-administered group (test group VI) (645,099 ± 557,005), and the GF-101 (10 U) + GF-203 administered group (test group VII) (780,577 ± 471,433) showed CTF values that were decreased by 37.5%, 12.1%, 49.6% and 39.0%, respectively. Furthermore, it was observed that the CNV lesions in the test group VI administered with GF-101 (20 U) and the test group VII administered with GF-101 (10 U) + GF-203 were significantly decreased compared to the CNV lesions in the PBS-administered group which was the control group (see FIG. 2).

### Optical Coherence Tomography (OCT)

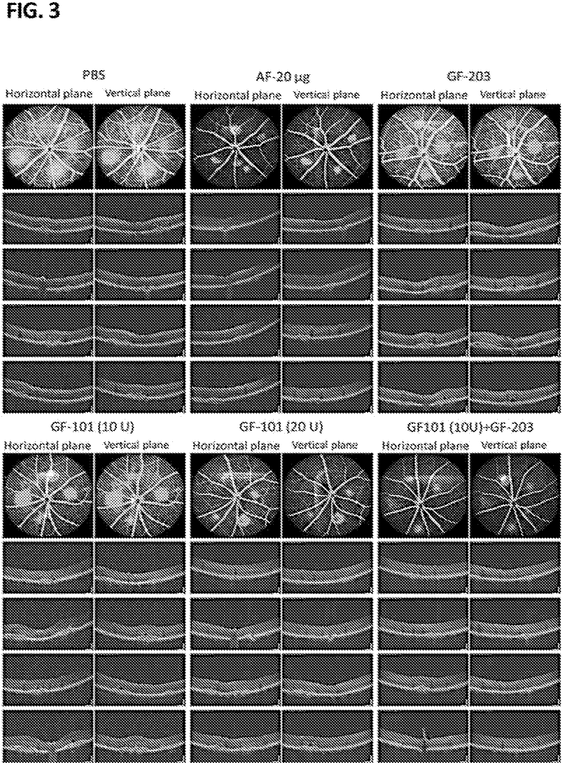

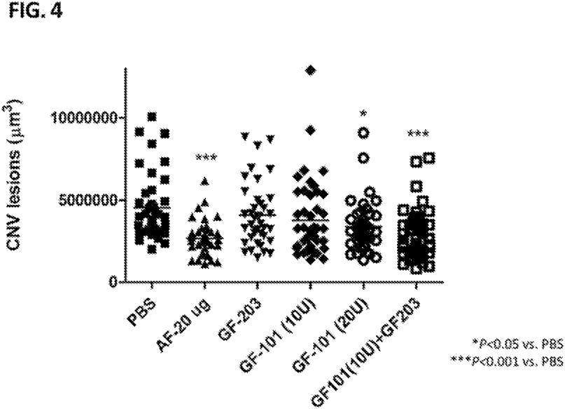

As shown in FIG. 1, on 12 days after laser irradiation, fundus fluorescein angiography imaging was performed, and at the same time, optical coherence tomography (OCT) was performed to obtain the detailed sections and 3D images of the eyes from the mouse retinas. Tomography of each lesion site was performed by transmitting an OCT beam through the center of the CNV lesion on the fundus fluorescein angiography image, and the image J program was used to quantify the CNV lesion. Retinal tomography was performed by changing the direction of the OCT beam horizontally and vertically for each laser bum. The size of the CNV lesions was measured, and the results are shown FIG. 3 and FIG. 4.

The eye retinal thickness of the mice, measured by optical coherence tomography (OCT), was decreased compared to the ocular retinal thickness measured before administration of the compositions of the present invention. Specifically, the size of the CNV lesions was 4,548,182 ± 1,983,055 µm³ in the PBS-administered group (test group II) and was 2,674,277 ± 1,064,973 µm³ in test group III (aflibercept-administered group), which decreased by 41.2% compared to that in the PBS-administered group (test group II). The GF-101 (20 U)-administered group (test group VI) (3,471,454 ± 1,534,395 µm³) showed CNV lesions that decreased by 23.6%, indicating that the CNV lesions were significantly decreased by the administration of GF-101 (20 U).

The GF-203-administered group (test group IV) (4,087,991 ± 1,933,522 µm³), the GF-101 (10 U)-administered group (test group V) (3,777,355 ± 2,302,834 µm³), the GF-101 (20 U)-administered group (test group VI) (3,471,454 ± 1,534,395 µm³) and the GF-101 (10 U) + GF203-administered group (test group VII) showed CNV lesions that decreased by 10.1%, 16.9%, 23.6% and 36.4%, respectively. Of the tested groups, groups III, VI and VII showed statistically significant decrease in CNV lesion.

### Electroretinography (ERG)

To evaluate a retinal function, mice were dark-adapted for 24 hours and subjected to electroretinography in the dark on 13 days after laser irradiation. Electroretinography measures the electrical activity produced by photoreceptor cells in the retina when the eye is stimulated by a specific light source. These measurements are recorded through electrodes disposed on the front surface of the eye (e.g., the cornea) and on the skin near the eye, thereby producing a graph called an electroretinogram (ERG).

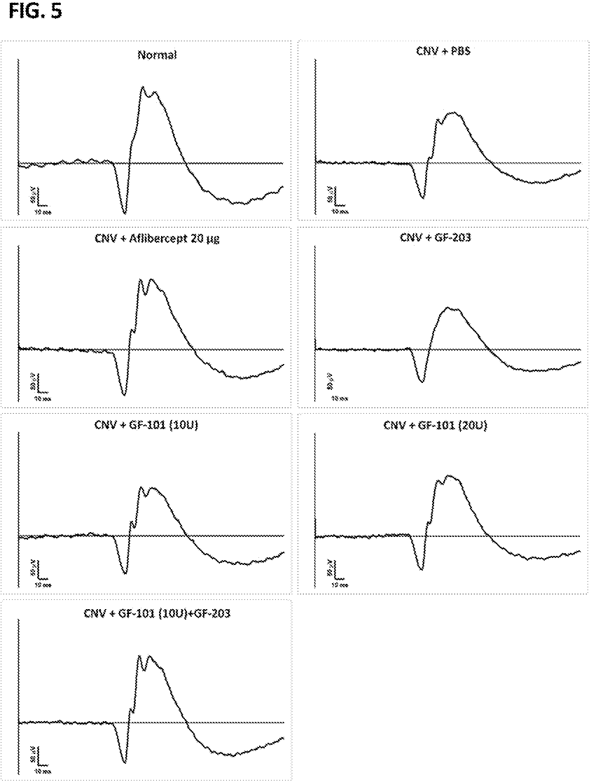

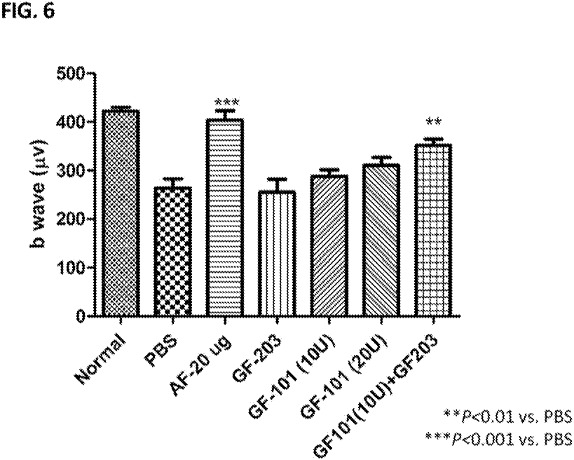

For electroretinography, both eyes of CNV mice were dilated and anesthetized, and then electroretinography was performed by bringing electrodes into contact with the skin, tail, and cornea, respectively. The retina was stimulated by a single white light with a flash intensity of 0.8 cd · sec/m² to obtain a response value. The amplitude was measured from the valley of the a-wave to the apex of the b-wave, and the results of the measurement are shown in FIG. 5 and FIG. 6. The amplitude was evaluated as an indicator of retinal function.

Referring to FIG. 5, the amplitude of the Scotopic b-wave was 263.64 ± 59.88 µV in test group II (PBS-administered group), which decreased by 153.13 µV compared to that of test group I (normal group) (422.27 ± 27.34 µV). The b-wave amplitude of test group III was 403.97 ± 53.79 µV, indicating that the responsiveness of this group was increased by the administration of aflibercept. However, in the case of the GF-203-administered group (test group IV) (255.25 ± 75.65 µV) and the GF-101 (10 U)-administered group (test group V) (288.233 ± 37.41 µV), the change in retinal function by drug administration could not be observed. The b-wave amplitude of the group administered with GF-101 (20 U) was 310.80 ± 53.42 µV, indicating that this group had increased responsiveness to light, but no statistical significance appeared. The b-wave amplitude of the group administered with the combination of GF-101 (10 U) and GF-203 (test group VII) was 351.62 ± 41.59 µV, which significantly increased compared to that of the negative control group.

### Statistical Analysis

The percentage of laser spots with CNV at different doses of a SOD or its 100 kD fragment derived from the B. amyloliquefaciens GF423 strain was compared pair-wise by a chi-square test. The results were plotted against the dose of the SOD derived from the B. amyloliquefaciens GF423 strain to derive the best-fit curve, which was used to calculate the dose of SOD that reduces the fraction of laser spots with CNV by 50% (ED₅₀). A confidence level of p<0.05 was considered statistically significant.

### 6.4. Histological Analysis

In order to observe the change in tissue by a laser, the mouse eyes were enucleated and fixed with 10% formalin for 10 minutes, and then they were placed in disposable base molds, embedded in an OCT compound, and frozen rapidly in liquid nitrogen.

### Hematoxylin & Eosin (H & E) Staining

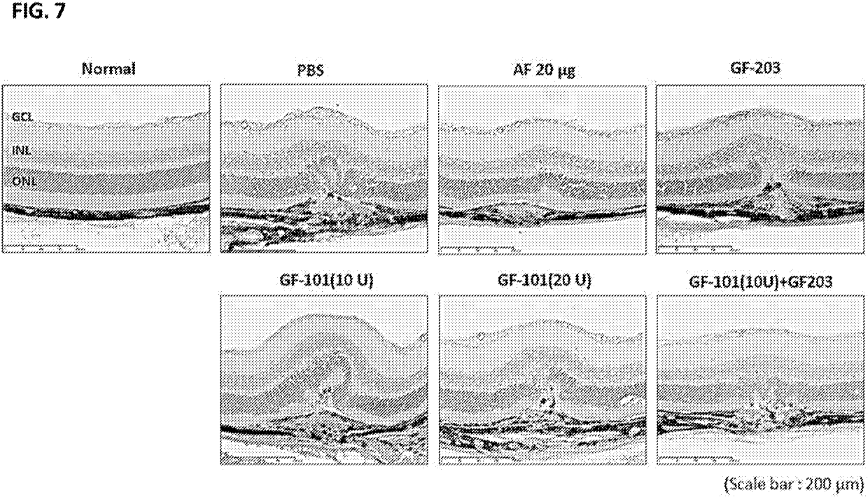

The tissue samples treated by the above-described method were sectioned, attached to slides, and then dried for about 1 hour, followed by the construction of CNV models. Then, in order to observe the changes in mouse retinas by drug treatment, the samples were stained with hematoxylin & eosin (H & E) and washed. The samples were treated with HCl solution and stained with eosin solution for 30 seconds to 1 minute, and then washed again. The samples were treated with 80%, 85%, 90% and 100% ethanol for 3 minutes for each treatment, and then reacted with carboxylene and xylene for 5 minutes for each reaction. Next, the embedded tissues were imaged with a virtual microscope (NanoZoomer 2.0 RS), and the images are shown in FIG. 7.

FIG. 7 shows choroidal neovascularization in the eyes (after H & E staining) of the laser-irradiated CNV mice compared to the normal group. In the group administered with PBS after CNV induction, CNV generation was observed together with tissue collapse of the laser-irradiated site. In the GF-203-administered group (test group IV) and the GF-101 (10 U)-administered group (test group V), the CNV lesions did not decrease significantly. However, the CNV lesions are decreased in the GF-101 (20 U)-administered group (test group VI) and the GF-101 (10 U) + GF-203-administered group (test group VII)

### TUNEL Assay

A TUNEL assay was performed to observe dead cells in the mouse retina after drug treatment in CNV models. Staining was performed using a fluorescence detection TUNEL assay kit. The tissue sections were de-paraffinized with xylene, and then hydrated twice with 100% ethanol, once with 95% ethanol and once with 85% ethanol in order, followed by washing once with PBS. The tissue surface was wiped clean, and the slides were incubated directly with proteinase K (20 µg/mL) at room temperature for 15 minutes, and then washed twice with PBS. The tissue surface was wiped clean and the slides were incubated directly with 75 µl of equilibration buffer at room temperature for 10 seconds. The tissue surface was wiped clean and the slides were incubated directly with 55 µl of working strength TdT enzyme 37° C. for 1 hour. The slides were washed by shaking with a working strength stop/wash buffer for 15 seconds and then incubated for 10 minutes at room temperature, followed by washing three times with PBS. The tissue surface was wiped clean, incubated directly with 65 µl of an anti-digoxigenin conjugate, and allowed to be left at room temperature for 30 minutes under light-shielded conditions. The slides were washed four times with PBS, stained with DAPI, and then observed with a fluorescence microscope (LEICA DM 2500).

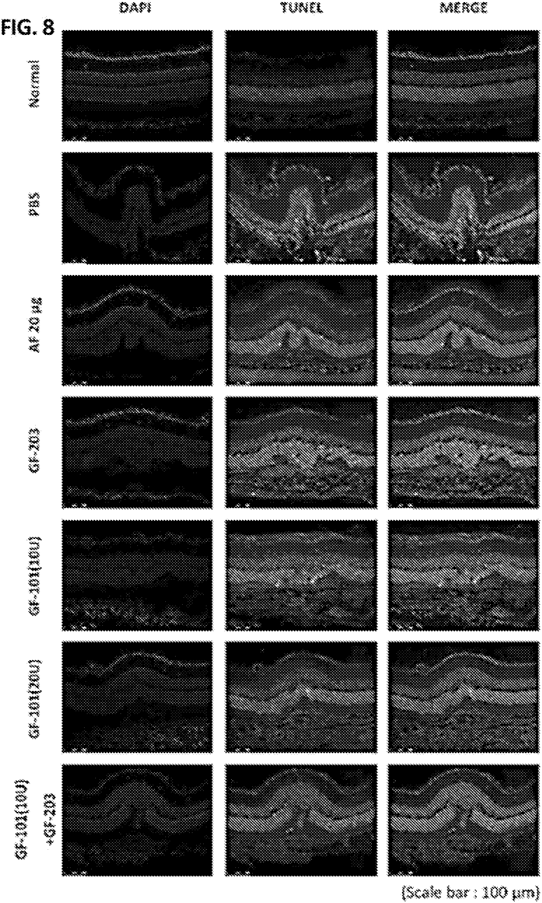

FIG. 8 shows a TUNEL assay performed to observe dead cells in the mouse retina after drug treatment in CNV models. TUNEL response indicative of cell death was observed intensively in the CNV site and in the outer nuclear layer (ONL). The highest number of dead cells was found in the group treated with PBS after CNV induction (test group II), and the number of dead cells in the GF-101 (20 U)-administered group (test group VI) and the group administered with the combination of GF-101 and GF-203 (test group VII) decreased to a level similar to that in the positive control aflibercept-administered group (test group III) (see FIG. 8).

### Immuno-Fluorescence Staining

Sections were permeabilized with 0.5% of Triton X-100 solution and washed three times with PBS (5 minutes for each wash). Sections were incubated with a blocking buffer (5% normal serum of the secondary antibody species (goat or donkey) including 3% BSA and 0.5% Triton X-100) for 1 hour followed by incubation with anti-VEGF and anti-STAT3 primary antibodies in PBS including 3% BSA and 0.5% Triton X-100 at 4° C. overnight.

Sections were washed with PBS three times for 5 minutes and incubated with a secondary antibody at a dilution of 1:1000 at room temperature for 1 hour. They were then washed with PBS three times (5 minutes for each wash). After staining with DAPI, they were mounted and observed under a fluorescent microscope (LEICA DM 2500)

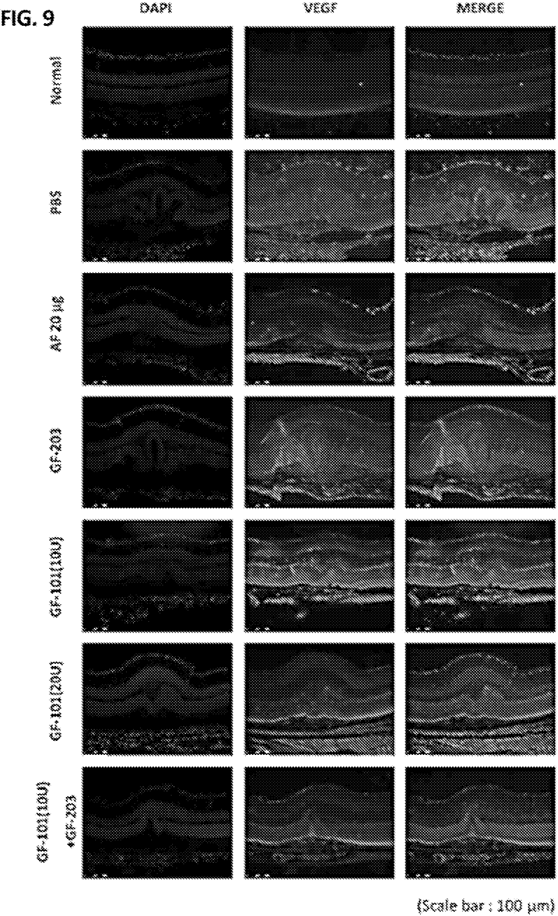

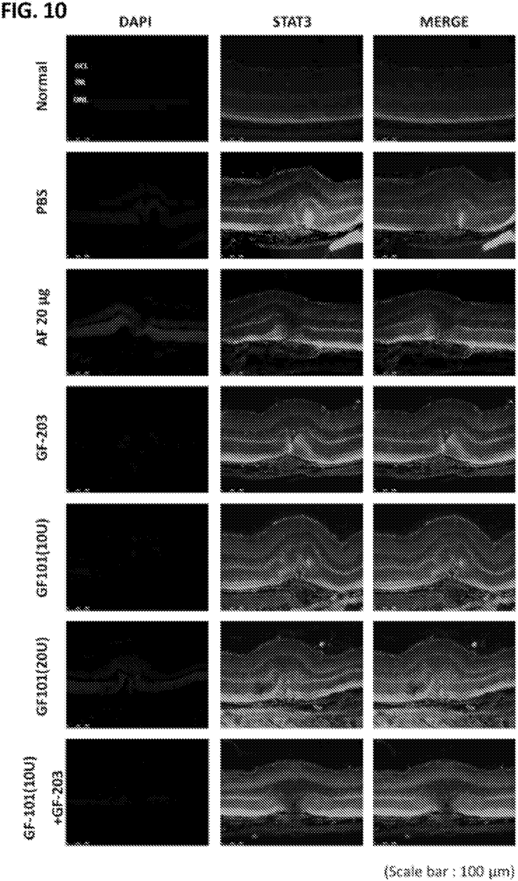

FIG. 9 and FIG. 10 show IF staining to observe the expression of VEGF and STAT3 in mouse retina after drug treatment in CNV models. In the PBS group, VEGF was highly expressed at the outer nuclear layer and CNV lesion. However, VEGF expression levels were decreased by aflibercept (group III), GF-101 (20 U) (group VI) and GF-101 (10 U) + GF203 (group VII) (FIG. 9). The STAT3 level in CNV lesion was also decreased in aflibercept (group III), GF-101 (20 U) (group VI) and GF-101 (10 U) + GF203 (group VII) groups (FIG. 10)

### Western Hybridization

Western blot assay was performed to measure the expression level of Nuclear factor erythroid 2-related factor 2 (NRF2) and hypoxia-inducible factor-1 alpha (HIF-1α) in response to treatment. The retina was homogenized and then the total protein was extracted with Pro-PREP (iNtRON Biotechnology, Korea). The protein concentration was measured by BCA protein assay kit (Thermo scientific, USA). Twenty µg of the protein was used for western hybridization. Signal was visualized by Gel documentation system (Fusion FX spectra). The Western signal of Nrf2 and HIF-1α were normalized with the signal of β-actin. Statistical analysis was performed by pairwise t-test and a confidence level of p<0.05 was considered statistically significant.

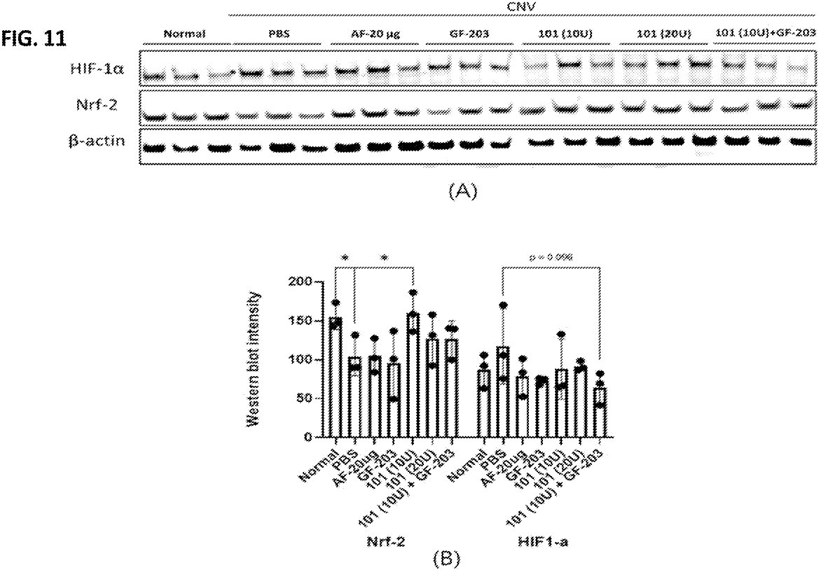

FIG. 11 shows the results of Western hybridization to observe the expression of HIF-1α and NRF2 in mouse retina after drug treatment in CNV models. In the PBS group, level of HIF-1α was increased and that of NRF2, decreased in retina compared to naive group. The level of NRF2 was increase in groups administered with GF101 (test groups V and VI) and the group administered with the combination of GF-101 and GF-203 (test group VII). The level of HIF-1α were decreased in all test groups. The most prominent decrease of HIF-1α level was observed in GF-101 (10 U) + GF203 (group VII) group.

### 6.5. Results

Blood vessels were stained with fluorescein and subjected to fundus fluorescein angiography. As a result, the GF-101 (20 U)-administered group (test group VI) and the group administered with the combination of GF-101 and GF-203 (test group VII) showed significantly low CTF values (FIG. 2).

The CNV lesions were also measured with OCT. The measurement of CNV lesion by OCT is considered to be more accurate than fluorescein angiography. The results of OCT showed a tendency to the results of fundus fluorescein angiography, but the efficacy was shown to be the highest in the group administered with the combination of GF-101 and GF-203 (FIG. 4), which showed relatively high CTF values in the fundus fluorescein angiography.

In electroretinography, the amplitude in the CNV-induced group (test group II) decreased by about 150 µV compared to that in the normal group (test group I), indicating that the retinal function of test group II was declined. The b-wave amplitude of the group administered with the combination of GF-101 and GF-203 showed statistically significant increase in responsiveness to light (FIG. 6) indicating the retinal function of the group VII was restored.

For histological analysis, CNV lesions were analyzed by H & E staining, and photoreceptor cell death in the CNV site was analyzed using TUNEL staining. Increasing CNV size affected the surrounding tissues, and cells damaged in this process were observed in the outer nuclear layer (ONL). However, fewer dead cells were observed in the GF-101 (20 U)-administered group (test group VI) and the group administered with the combination of GF-101 and GF-203 (test group VII) (FIG. 8.). VEGF, a representative angiogenesis factor, is known to have a direct effect on the formation of choroidal neovascularization. Strong VEGF expression could be observed in the CNV region formed by laser irradiation, and this expression was most effectively inhibited in the GF-101 (20 U)-administered group (test group VI) and the group administered with the combination of GF-101 and GF-203 (test group VII).

In summary, it is demonstrated herein that the combination of GF-101 and GF-203 restored retinal function by effectively suppressing the choroidal neovascularization induced by laser irradiation, as demonstrated by decrease in CNV lesion and VEGF expression. The combination of GF-101 and GF-203 was more effective than GF-101 in reducing of CNV lesion, judged by observation with OCT, restoring retinal function, judged by ERG and inhibiting HIF-1α expression, judged by Western hybridization.

The compositions of the present disclosure, comprising SOD derived from B. amyloliquefaciens GF423 strain, have excellent antioxidant activity, highly stable enzyme activity, and excellent in vivo stability, and thus can be advantageously used as a material for a pharmaceutical drug, a food, a medical food, etc. for preventing or treating macular degeneration, particularly age-related macular degeneration.

The description provided herein is illustrative of preferred embodiments and is not intended to limit the scope of the present invention. It will be obvious to those skilled in the art that various modifications and changes are possible without departing from the spirit and scope of the present invention.

### [Accession Numbers]

Depository authority: the Korea Research Institute of Bioscience and Biotechnology
KCTC 13222BP
Deposit date: Mar. 6, 2017
KCTC 13227BP
Deposit date: Mar. 23, 2017

TABLE 3

Representative Sequences
SEQ ID NO: 1 SOD enzyme amino acid sequence (Bacillus amyloliquefaciens)
The SOD enzyme from GF-423 and the SOD enzyme from GF-424 have the same amino acid sequence presented below.

| | | | | | | | | | | | | | | | |
|---|---|---|---|---|---|---|---|---|---|---|---|---|---|---|---|
| Met | Ala | Tyr | Lys | Leu | Pro | Glu | Leu | Pro | Tyr | Ala | Tyr | Asp | Ala | Leu | Glu |
| 1 | | | | 5 | | | | | 10 | | | | | 15 | |
| Pro | His | Ile | Asp | Lys | Glu | Thr | Met | Thr | Ile | His | His | Thr | Lys | His | His |
| | | | 20 | | | | | 25 | | | | | 30 | | |
| Asn | Thr | Tyr | Val | Thr | Asn | Leu | Asn | Lys | Ala | Ile | Glu | Gly | Ser | Ala | Leu |
| | | 35 | | | | | 40 | | | | | 45 | | | |
| Ala | Glu | Lys | Ser | Val | Asp | Glu | Leu | Val | Ala | Asp | Leu | Asn | Ala | Val | Pro |
| | 50 | | | | | 55 | | | | | 60 | | | | |
| Glu | Asp | Ile | Arg | Thr | Ala | Val | Arg | Asn | Asn | Gly | Gly | Gly | His | Ala | Asn |
| 65 | | | | | 70 | | | | | 75 | | | | | 80 |
| His | Ser | Leu | Phe | Trp | Thr | Leu | Leu | Ser | Pro | Asn | Gly | Gly | Gly | Glu | Pro |
| | | | | 85 | | | | | 90 | | | | | 95 | |
| Thr | Gly | Glu | Leu | Ala | Glu | Glu | Ile | Lys | Ser | Thr | Phe | Gly | Ser | Phe | Asp |
| | | | 100 | | | | | 105 | | | | | 110 | | |
| Gln | Phe | Lys | Glu | Lys | Phe | Ala | Ala | Ala | Ala | Ala | Gly | Arg | Phe | Gly | Ser |
| | | 115 | | | | | 120 | | | | | 125 | | | |
| Gly | Trp | Ala | Trp | Leu | Val | Val | Asn | Asn | Gly | Lys | Leu | Glu | Ile | Thr | Ser |
| | 130 | | | | | 135 | | | | | 140 | | | | |
| Thr | Pro | Asn | Gln | Asp | Ser | Pro | Leu | Ser | Glu | Gly | Lys | Thr | Pro | Val | Leu |
| 145 | | | | | 150 | | | | | 155 | | | | | 160 |
| Gly | Leu | Asp | Val | Trp | Glu | His | Ala | Tyr | Tyr | Leu | Asn | Tyr | Gln | Asn | Arg |
| | | | | 165 | | | | | 170 | | | | | 175 | |
| Arg | Pro | Asp | Tyr | Ile | Ser | Ala | Phe | Trp | Asn | Val | Val | Asn | Trp | Asp | Glu |
| | | | 180 | | | | | 185 | | | | | 190 | | |
| Val | Ala | Arg | Leu | Tyr | Ser | Glu | Ala | Lys | | | | | | | |

SEQ ID NO: 2 (Bacillus amyloliquefaciens)

| | |
|---|---|
| agagtttgat cctggctcag gacgaacgct ggcggcgtgc ctaatacatg caagtcgagc | 60 |
| ggacagatgg gagcttgctc cctgatgtca gcggcggacg ggtgagtaac acgtgggtaa | 120 |
| cctgcctgta agactgggat aactccggga aaccggggct aataccggat ggttgtttga | 180 |
| accgcatggt tcagacataa aaggtggctt cggctaccac ttacagatgg acccgcggcg | 240 |
| cattagctag ttggtgaggt aacggctcac caaggcaacg atgcgtagcc gacctgagag | 300 |
| ggtgatcggc cacactggga ctgagacacg gcccagactc ctacgggagg cagcagtagg | 360 |
| gaatcttccg caatggacga aagtctgacg gagcaacgcc gcgtgagtga tgaaggtttt | 420 |
| cggatcgtaa agctctgttg ttagggaaga acaagtgccg ttcaaatagg gcggcacctt | 480 |
| gacggtacct aaccagaaag ccacggctaa ctacgtgcca gcagccgcgg taatacgtag | 540 |
| gtggcaagcg ttgtccggaa ttattgggcg taaagggctc gcaggcggtt tcttaagtct | 600 |
| gatgtgaaag cccccggctc aaccggggag ggtcattgga aactggggaa cttgagtgca | 660 |
| gaagaggaga gtggaattcc acgtgtagcg gtgaaatgcg tagagatgtg gaggaacacc | 720 |
| agtggcgaag gcgactctct ggtctgtaac tgacgctgag gagcgaaagc gtggggagcg | 780 |
| aacaggatta gataccctgg tagtccacgc cgtaaacgat gagtgctaag tgttaggggg | 840 |
| tttccgcccc ttagtgctgc agctaacgca ttaagcactc cgcctgggga gtacggtcgc | 900 |
| aagactgaaa ctcaaaggaa ttgacggggg cccgcacaag cggtggagca tgtggtttaa | 960 |
| ttcgaagcaa cgcgaagaac cttaccaggt cttgacatcc tctgacaatc ctagagatag | 1020 |
| gacgtcccct tcgggggcag agtgacaggt ggtgcatggt tgtcgtcagc tcgtgtcgtg | 1080 |
| agatgttggg ttaagtcccg caacgagcgc aacccttgat cttagttgcc agcattcagt | 1140 |
| tgggcactct aaggtgactg ccggtgacaa accggaggaa ggtggggatg acgtcaaatc | 1200 |
| atcatgcccc ttatgacctg ggctacacac gtgctacaat ggacagaaca aagggcagcg | 1260 |
| aaaccgcgag gttaagccaa tcccacaaat ctgttctcag ttcggatcgc agtctgcaac | 1320 |
| tcgactgcgt gaagctggaa tcgctagtaa tcgcggatca gcatgccgcg gtgaatacgt | 1380 |
| tcccgggcct tgtacacacc gcccgtcaca ccacgagagt ttgtaacacc cgaagtcggt | 1440 |
| gaggtaacct ttatggagcc agccgccgaa ggtgggacag atgattgggg tgaagtcgta | 1500 |
| acaaggtagc cgtatcggaa ggtgcggctg gatcacctcc ttt | 1543 |

SEQ ID NO: 3 (Bacillus amyloliquefaciens)

| | |
|---|---|
| agagtttgat cctggctcag gacgaacgct ggcggcgtgc ctaatacatg caagtcgagc | 60 |
| ggacagatgg gagcttgctc cctgatgtta gcggcggacg ggtgagtaac acgtgggtaa | 120 |
| cctgcctgta agactgggat aactccggga aaccggggct aatagcggat ggttgtttga | 180 |
| accgcatggt tcagacataa aaggtggctt cggctaccac ttacagatgg acccgcggcg | 240 |
| cattagctag ttggtgaggt aacggctcac caaggcgacg atgcgtagcc gacctgagag | 300 |
| ggtgatcggc cacactggga ctgagacacg gcccagactc ctacgggagg cagcagtagg | 360 |
| gaatcttccg caatggacga aagtctgacg gagcaacgcc gcgtgagtga tgaaggtttt | 420 |
| cggatcgtaa agctctgttg ttagggaaga acaagtgccg ttcaaatagg gcggcacctt | 480 |
| gacggtacct aaccagaaag ccacggctaa ctacgtgcca gcagccgcgg taatacgtag | 540 |
| gtggcaagcg ttgtccggaa ttattgggcg taaagggctc gcaggcggtt tcttaagtct | 600 |
| gatgtgaaag cccccggctc aaccggggag ggtcattgga aactggggaa cttgagtgca | 660 |
| gaagaggaga gtggaattcc acgtgtagcg gtgaaatgcg tagagatgtg gaggaacacc | 720 |
| agtggcgaag gcgactctct ggtctgtaac tgacgctgag gagcgaaagc gtggggagcg | 780 |
| aacaggatta gataccctgg tagtccacgc cgtaaacgat gagtgctaag tgttaggggg | 840 |
| tttccgcccc ttagtgctgc agctaacgca ttaagcactc cgcctgggga gtacggtcgc | 900 |
| aagactgaaa ctcaaaggaa ttgacggggg cccgcacaag cggtggagca tgtggtttaa | 960 |
| ttcgaagcaa cgcgaagaac cttaccaggt cttgacatcc tctgacaatc ctagagatag | 1020 |
| gacgtcccct tcgggggcag agtgacaggt ggtgcatggt tgtcgtcagc tcgtgtcgtg | 1080 |
| agatgttggg ttaagtcccg caacgagcgc aacccttgat cttagttgcc agcattcagt | 1140 |
| tgggcactct aaggtgactg ccggtgacaa accggaggaa ggtggggatg acgtcaaatc | 1200 |
| atcatgcccc ttatgacctg ggctacacac gtgctacaat ggacagaaca aagggcagcg | 1260 |
| aaaccgcgag gttaagccaa tcccacaaat ctgttctcag ttcggatcgc agtctgcaac | 1320 |
| tcgactgcgt gaagctggaa tcgctagtaa tcgcggatca gcatgccgcg gtgaatacgt | 1380 |
| tcccgggcct tgtacacacc gcccgtcaca ccacgagagt ttgtaacacc cgaagtcggt | 1440 |
| gaggtaacct ttatggagcc agccgccgaa ggtgggacag atgattgggg tgaagtcgta | 1500 |
| acaaggtagc cgtatcggaa ggtgcggctg gatcacctcc ttt | 1543 |

SEQ ID NO: 4 (Bacillus amyloliquefaciens)

| | |
|---|---|
| agagtttgat cctggctcag gacgaacgct ggcggcgtgc ctaatacatg caagtcgagc | 60 |
| ggacagatgg gagcttgctc cctgatgtta gcggcggacg ggtgagtaac acgtgggtaa | 120 |
| cctgcctgta agactgggat aactccggga aaccggggct aataccggat ggttgtctga | 180 |
| accgcatggt tcagacataa aaggtggctt cggctaccac ttacagatgg acccgcggcg | 240 |
| cattagctag ttggtgaggt aacggctcac caaggcgacg atgcgtagcc gacctgagag | 300 |
| ggtgatcggc cacactggga ctgagacacg gcccagactc ctacgggagg cagcagtagg | 360 |
| gaatcttccg caatggacga aagtctgacg gagcaacgcc gcgtgagtga tgaaggtttt | 420 |
| cggatcgtaa agctctgttg ttagggaaga acaagtgccg ttcaaatagg gcggcacctt | 480 |
| gacggtacct aaccagaaag ccacggctaa ctacgtgcca gcagccgcgg taatacgtag | 540 |
| gtggcaagcg ttgtccggaa ttattgggcg taaagggctc gcaggcggtt tcttaagtct | 600 |
| gatgtgaaag cccccggctc aaccggggag ggtcattgga aactggggaa cttgagtgca | 660 |
| gaagaggaga gtggaattcc acgtgtagcg gtgaaatgcg tagagatgtg gaggaacacc | 720 |
| agtggcgaag gcgactctct ggtctgtaac tgacgctgag gagcgaaagc gtggggagcg | 780 |
| aacaggatta gataccctgg tagtccacgc cgtaaacgat gagtgctaag tgttaggggg | 840 |
| tttccgcccc ttagtgctgc agctaacgca ttaagcactc cgcctgggga gtacggtcgc | 900 |
| aagactgaaa ctcaaaggaa ttgacggggg cccgcacaag cggtggagca tgtggtttaa | 960 |
| ttcgaagcaa cgcgaagaac cttaccaggt cttgacatcc tctgacaatc ctagagatag | 1020 |
| gacgtcccct tcgggggcag agtgacaggt ggtgcatggt tgtcgtcagc tcgtgtcgtg | 1080 |
| agatgttggg ttaagtcccg caacgagcgc aacccttgat cttagttgcc agcattcagt | 1140 |
| tgggcactct aaggtgactg ccggtgacaa accggaggaa ggtggggatg acgtcaaatc | 1200 |
| atcatgcccc ttatgacctg ggctacacac gtgctacaat ggacagaaca aagggcagcg | 1260 |
| aaaccgcgag gttaagccaa tcccacaaat ctgttctcag ttcggatcgc agtctgcaac | 1320 |
| tcgactgcgt gaagctggaa tcgctagtaa tcgcggatca gcatgccgcg gtgaatacgt | 1380 |
| tcccgggcct tgtacacacc gcccgtcaca ccacgagagt ttgtaacacc cgaagtcggt | 1440 |
| gaggtaacct ttatggagcc agccgccgaa ggtgggacag atgattgggg tgaagtcgta | 1500 |
| acaaggtagc cgtatcggaa ggtgcggctg gatcacctcc ttt | 1543 |

SEQ ID NO: 5 (Bacillus amyloliquefaciens)

| | |
|---|---|
| agagtttgat cctggctcag gacgaacgct ggcggcgtgc ctaatacatg caagtcgagc | 60 |
| ggacagatgg gagcttgctc cctgatgtta gcggcggacg ggtgagtaac acgtgggtaa | 120 |
| cctgcctgta agactgggat aactccggga aaccggggct aataccggat ggttgtttga | 180 |
| accgcatggt tcagacataa aaggtggctt cggctaccac ttacagatgg acccgcggcg | 240 |
| cattagctag ttggtgaggt aacggctcac caaggcgacg atgcgtagcc gacctgagag | 300 |
| ggtgatcggc cacactggga ctgagacacg gcccagactc ctacgggagg cagcagtagg | 360 |
| gaatcttccg caatggacga aagtctgacg gagcaacgcc gcgtgagtga tgaaggtttt | 420 |
| cggatcgtaa agctctgttg ttagggaaga acaagtgccg ttcaaatagg gcggcacctt | 480 |
| gacggtacct aaccagaaag ccacggctaa ctacgtgcca gcagccgcgg taatacgtag | 540 |
| gtggcaagcg ttgtccggaa ttattgggcg taaagggctc gcaggcggtt tcttaagtct | 600 |
| gatgtgaaag cccccggctc aaccggggag ggtcattgga aactggggaa cttgagtgca | 660 |
| gaagaggaga gtggaattcc acgtgtagcg gtgaaatgcg tagagatgtg gaggaacacc | 720 |
| agtggcgaag gcgactctct ggtctgtaac tgacgctgag gagcgaaagc gtggggagcg | 780 |
| aacaggatta gataccctgg tagtccacgc cgtaaacgat gagtgctaag tgttaggggg | 840 |
| tttccgcccc ttagtgctgc agctaacgca ttaagcactc cgcctgggga gtacggtcgc | 900 |
| aagactgaaa ctcaaaggaa ttgacggggg cccgcacaag cggtggagca tgtggtttaa | 960 |
| ttcgaagcaa cgcgaagaac cttaccaggt cttgacatcc tctgacaatc ctagagatag | 1020 |
| gacgtcccct tcgggggcag agtgacaggt ggtgcatggt tgtcgtcagc tcgtgtcgtg | 1080 |
| agatgttggg ttaagtcccg caacgagcgc aacccttgat cttagttgcc agcattcagt | 1140 |
| tgggcactct aaggtgactg ccggtgacaa accggaggaa ggtggggatg acgtcaaatc | 1200 |
| atcatgcccc ttatgacctg ggctacacac gtgctacaat ggacagaaca aagggcagcg | 1260 |
| aaaccgcgag gttaagccaa tcccacaaat ctgttctcag ttcggatcgc agtctgcaac | 1320 |
| tcgactgcgt gaagctggaa tcgctagtaa tcgcggatca gcatgccgcg gtgaatacgt | 1380 |
| tcccgggcct tgtacacacc gcccgtcaca ccacgagagt ttgtaacacc cgaagtcggt | 1440 |
| gaggtaacct ttatggagcc agccgccgaa ggtgggacag atgattgggg tgaagtcgta | 1500 |
| acaaggtagc cgtatcggaa ggtgcggctg gatcacctcc ttt | 1543 |

SEQ ID NO: 6 (Bacillus amyloliquefaciens)

| | |
|---|---|
| agagtttgat cctggctcag gacgaacgct ggcggcgtgc ctaatacatg caagtcgagc | 60 |
| ggacagatgg gagcttgctc cctgatgtta gcggcggacg ggtgagtaac acgtgggtaa | 120 |
| cctgcctgta agactgggat aactccggga aaccggggct aataccggat ggttgtctga | 180 |
| accgcatggt tcagacataa aaggtggctt cggctaccac ttacagatgg acccgcggcg | 240 |
| cattagctag ttggtgaggt aacggctcac caaggcgacg atgcgtagcc gacctgagag | 300 |
| ggtgatcggc cacactggga ctgagacacg gcccagactc ctacgggagg cagcagtagg | 360 |
| gaatcttccg caatggacga aagtctgacg gagcaacgcc gcgtgagtga tgaaggtttt | 420 |
| cggatcgtaa agctctgttg ttagggaaga acaagtgccg ttcaaatagg gcggcacctt | 480 |
| gacggtacct aaccagaaag ccacggctaa ctacgtgcca gcagccgcgg taatacgtag | 540 |
| gtggcaagcg ttgtccggaa ttattgggcg taaagggctc gcaggcggtt tcttaagtct | 600 |
| gatgtgaaag cccccggctc aaccggggag ggtcattgga aactggggaa cttgagtgca | 660 |
| gaagaggaga gtggaattcc acgtgtagcg gtgaaatgcg tagagatgtg gaggaacacc | 720 |
| agtggcgaag gcgactctct ggtctgtaac tgacgctgag gagcgaaagc gtggggagcg | 780 |
| aacaggatta gataccctgg tagtccacgc cgtaaacgat gagtgctaag tgttaggggg | 840 |
| tttccgcccc ttagtgctgc agctaacgca ttaagcactc cgcctgggga gtacggtcgc | 900 |
| aagactgaaa ctcaaaggaa ttgacggggg cccgcacaag cggtggagca tgtggtttaa | 960 |
| ttcgaagcaa cgcgaagaac cttaccaggt cttgacatcc tctgacaatc ctagagatag | 1020 |
| gatgtcccct tcgggggcag agtgacaggt ggtgcatggt tgtcgtcagc tcgtgtcgtg | 1080 |
| agatgttggg ttaagtcccg caacgagcgc aacccttgat cttagttgcc agcattcagt | 1140 |
| tgggcactct aaggtgactg ccggtgacaa accggaggaa ggtggggatg acgtcaaatc | 1200 |
| atcatgcccc ttatgacctg ggctacacac gtgctacaat ggacagaaca aagggcagcg | 1260 |
| aaaccgcgag gttaagccaa tcccacaaat ctgttctcag ttcggatcgc agtctgcaac | 1320 |
| tcgactgcgt gaagctggaa tcgctagtaa tcgcggatca gcatgccgcg gtgaatacgt | 1380 |
| tcccgggcct tgtacacacc gcccgtcaca ccacgagagt ttgtaacacc cgaagtcggt | 1440 |
| gaggtaacct ttatggagcc agccgccgaa ggtgggacag atgattgggg tgaagtcgta | 1500 |
| acaaggtagc cgtatcggaa ggtgcggctg gatcacctcc ttt | 1543 |

SEQ ID NO: 7 (Bacillus amyloliquefaciens)

| | |
|---|---|
| agagtttgat cctggctcag gacgaacgct ggcggcgtgc ctaatacatg caagtcgagc | 60 |
| ggacagatgg gagcttgctc cctgatgtta gcggcggacg ggtgagtaac acgtgggtaa | 120 |
| cctgcctgta agactgggat aactccggga aaccggggct aataccggat ggttgtctga | 180 |
| accgcatggt tcagacataa aaggtggctt cggctaccac ttacagatgg acccgcggcg | 240 |
| cattagctag ttggtgaggt aacggctcac caaggcgacg atgcgtagcc gacctgagag | 300 |
| ggtgatcggc cacactggga ctgagacacg gcccagactc ctacgggagg cagcagtagg | 360 |
| gaatcttccg caatggacga aagtctgacg gagcaacgcc gcgtgagtga tgaaggtttt | 420 |
| cggatcgtaa agctctgttg ttagggaaga acaagtgccg ttcaaatagg gcggcacctt | 480 |
| gacggtacct aaccagaaag ccacggctaa ctacgtgcca gcagccgcgg taatacgtag | 540 |
| gtggcaagcg ttgtccggaa ttattgggcg taaagggctc gcaggcggtt tcttaagtct | 600 |
| gatgtgaaag cccccggctc aaccggggag ggtcattgga aactggggaa cttgagtgca | 660 |
| gaagaggaga gtggaattcc acgtgtagcg gtgaaatgcg tagagatgtg gaggaacacc | 720 |
| agtggcgaag gcgactctct ggtctgtaac tgacgctgag gagcgaaagc gtggggagcg | 780 |
| aacaggatta gataccctgg tagtccacgc cgtaaacgat gagtgctaag tgttaggggg | 840 |
| tttccgcccc ttagtgctgc agctaacgca ttaagcactc cgcctgggga gtacggtcgc | 900 |
| aagactgaaa ctcaaaggaa ttgacggggg cccgcacaag cggtggagca tgtggtttaa | 960 |
| ttcgaagcaa cgcgaagaac cttaccaggt cttgacatcc tctgacaatc ctagagatag | 1020 |
| gatgtcccct tcgggggcag agtgacaggt ggtgcatggt tgtcgtcagc tcgtgtcgtg | 1080 |
| agatgttggg ttaagtcccg caacgagcgc aacccttgat cttagttgcc agcattcagt | 1140 |
| tgggcactct aaggtgactg ccggtgacaa accggaggaa ggtggggatg acgtcaaatc | 1200 |
| atcatgcccc ttatgacctg ggctacacac gtgctacaat ggacagaaca aagggcagcg | 1260 |
| aaaccgcgag gttaagccaa tcccacaaat ctgttctcag ttcggatcgc agtctgcaac | 1320 |
| tcgactgcgt gaagctggaa tcgctagtaa tcgcggatca gcatgccgcg gtgaatacgt | 1380 |
| tcccgggcct tgtacacacc gcccgtcaca ccacgagagt ttgtaacacc cgaagtcggt | 1440 |
| gaggtaacct ttatggagcc agccgccgaa ggtgggacag atgattgggg tgaagtcgta | 1500 |
| acaaggtagc cgtatcggaa ggtgcggctg gatcacctcc ttt | 1543 |

SEQ ID NO: 8 (Bacillus amyloliquefaciens)

| | |
|---|---|
| agagtttgat cctggctcag gacgaacgct ggcggcgtgc ctaatacatg caagtcgagc | 60 |
| ggacagatgg gagcttgctc cctgatgtta gcggcggacg ggtgagtaac acgtgggtaa | 120 |
| cctgcctgta agactgggat aactccggga aaccggggct aataccggat ggttgtttga | 180 |
| accgcatggt tcagacataa aaggtggctt cggctaccac ttacagatgg acccgcggcg | 240 |
| cattagctag ttggtgaggt aacggctcac caaggcaacg atgcgtagcc gacctgagag | 300 |
| ggtgatcggc cacactggga ctgagacacg gcccagactc ctacgggagg cagcagtagg | 360 |
| gaatcttccg caatggacga aagtctgacg gagcaacgcc gcgtgagtga tgaaggtttt | 420 |
| cggatcgtaa agctctgttg ttagggaaga acaagtgccg ttcaaatagg gcggcacctt | 480 |
| gacggtacct aaccagaaag ccacggctaa ctacgtgcca gcagccgcgg taatacgtag | 540 |
| gtggcaagcg ttgtccggaa ttattgggcg taaagggctc gcaggcggtt tcttaagtct | 600 |
| gatgtgaaag cccccggctc aaccggggag ggtcattgga aactggggaa cttgagtgca | 660 |
| gaagaggaga gtggaattcc acgtgtagcg gtgaaatgcg tagagatgtg gaggaacacc | 720 |
| agtggcgaag gcgactctct ggtctgtaac tgacgctgag gagcgaaagc gtggggagcg | 780 |
| aacaggatta gataccctgg tagtccacgc cgtaaacgat gagtgctaag tgttaggggg | 840 |
| tttccgcccc ttagtgctgc agctaacgca ttaagcactc cgcctgggga gtacggtcgc | 900 |
| aagactgaaa ctcaaaggaa ttgacggggg cccgcacaag cggtggagca tgtggtttaa | 960 |
| ttcgaagcaa cgcgaagaac cttaccaggt cttgacatcc tctgacaatc ctagagatag | 1020 |
| gacgtcccct tcgggggcag agtgacaggt ggtgcatggt tgtcgtcagc tcgtgtcgtg | 1080 |
| agatgttggg ttaagtcccg caacgagcgc aacccttgat cttagttgcc agcattcagt | 1140 |
| tgggcactct aaggtgactg ccggtgacaa accggaggaa ggtggggatg acgtcaaatc | 1200 |
| atcatgcccc ttatgacctg ggctacacac gtgctacaat ggacagaaca aagggcagcg | 1260 |
| aaaccgcgag gttaagccaa tcccacaaat ctgttctcag ttcggatcgc agtctgcaac | 1320 |
| tcgactgcgt gaagctggaa tcgctagtaa tcgcggatca gcatgccgcg gtgaatacgt | 1380 |
| tcccgggcct tgtacacacc gcccgtcaca ccacgagagt ttgtaacacc cgaagtcggt | 1440 |
| gaggtaacct ttatggagcc agccgccgaa ggtgggacag atgattgggg tgaagtcgta | 1500 |
| acaaggtagc cgtatcggaa ggtgcggctg gatcacctcc ttt | 1543 |

SEQ ID NO: 9 (Bacillus amyloliquefaciens)

| | |
|---|---|
| agagtttgat cctggctcag gacgaacgct ggcggcgtgc ctaatacatg caagtcgagc | 60 |
| ggacagatgg gagcttgctc cctgatgtta gcggcggacg ggtgagtaac acgtgggtaa | 120 |
| cctgcctgta agactgggat aactccggga aaccggggct aataccggat ggttgtctga | 180 |
| atcgcatggt tcagacataa aaggtggctt ctgctaccac ttacagatgg acccgcggcg | 240 |
| cattagctag ttggtgaggt aacggctcac caaggcgacg atgcgtagcc gacctgagag | 300 |
| ggtgatcggc cacactggga ctgagacacg gcccagactc ctacgggagg cagcagtagg | 360 |
| gaatcttccg caatggacga aagtctgacg gagcaacgcc gcgtgagtga tgaaggtttt | 420 |
| cggatcgtaa agctctgttg ttagggaaga acaagtgccg ttcaaatagg gcggcacctt | 480 |
| gacggtacct aaccagaaag ccacggctaa ctacgtgcca gcagccgcgg taatacgtag | 540 |
| gtggcaagcg ttgtccggaa ttattgggcg taaagggctc gcaggcggtt tcttaagtct | 600 |
| gatgtgaaag cccccggctc aaccggggag ggtcattgga aactggggaa cttgagtgca | 660 |
| gaagaggaga gtggaattcc acgtgtagcg gtgaaatgcg tagagatgtg gaggaacacc | 720 |
| agtggcgaag gcgactctct ggtctgtaac tgacgctgag gagcgaaagc gtggggagcg | 780 |
| aacaggatta gataccctgg tagtccacgc cgtaaacgat gagtgctaag tgttaggggg | 840 |
| tttccgcccc ttagtgctgc agctaacgca ttaagcactc cgcctgggga gtacggtcgc | 900 |
| aagactgaaa ctcaaaggaa ttgacggggg cccgcacaag cggtggagca tgtggtttaa | 960 |
| ttcgaagcaa cgcgaagaac cttaccaggt cttgacatcc tctgacaatc ctagagatag | 1020 |
| gacgtcccct tcgggggcag agtgacaggt ggtgcatggt tgtcgtcagc tcgtgtcgtg | 1080 |
| agatgttggg ttaagtcccg caacgagcgc aacccttgat cttagttgcc agcattcagt | 1140 |
| tgggcactct aaggtgactg ccggtgacaa accggaggaa ggtggggatg acgtcaaatc | 1200 |
| atcatgcccc ttatgacctg ggctacacac gtgctacaat ggacagaaca aagggcagcg | 1260 |
| aaaccgcgag gttaagccaa tcccacaaat ctgttctcag ttcggatcgc agtctgcaac | 1320 |
| tcgactgcgt gaagctggaa tcgctagtaa tcgcggatca gcatgccgcg gtgaatacgt | 1380 |
| tcccgggcct tgtacacacc gcccgtcaca ccacgagagt ttgtaacacc cgaagtcggt | 1440 |
| gaggtaacct tttaggagcc agccgccgaa ggtgggacag atgattgggg tgaagtcgta | 1500 |
| acaaggtagc cgtatcggaa ggtgcggctg gatcacctcc ttt | 1543 |

SEQ ID NO: 10 (Bacillus amyloliquefaciens)

| | |
|---|---|
| agagtttgat cctggctcag gacgaacgct ggcggcgtgc ctaatacatg caagtcgagc | 60 |
| ggacagatgg gagcttgctc cctgatgtta gcggcggacg ggtgagtaac acgtgggtaa | 120 |
| cctgcctgta agactgggat aactccggga aaccggggct aataccagat ggttgtctga | 180 |
| accgcatggt tcagacataa aaggtggctt cggctaccac ttacagatgg acccgcggcg | 240 |
| cattagctag ttggtgaggt aacggctcac caaggcgacg atgcgtagcc gacctgagag | 300 |
| ggtgatcggc cacactggga ctgagacacg gcccagactc ctacgggagg cagcagtagg | 360 |
| gaatcttccg caatggacga aagtctgacg gagcaacgcc gcgtgagtga tgaaggtttt | 420 |
| cggatcgtaa agctctgttg ttagggaaga acaagtgccg ttcaaatagg gcggcacctt | 480 |
| gacggtacct aaccagaaag ccacggctaa ctacgtgcca gcagccgcgg taatacgtag | 540 |
| gtggcaagcg ttgtccggaa ttattgggcg taaagggctc gcaggcggtt tcttaagtct | 600 |
| gatgtgaaag cccccggctc aaccggggag ggtcattgga aactggggaa cttgagtgca | 660 |
| gaagaggaga gtggaattcc acgtgtagcg gtgaaatgcg tagagatgtg gaggaacacc | 720 |
| agtggcgaag gcgactctct ggtctgtaac tgacgctgag gagcgaaagc gtggggagcg | 780 |
| aacaggatta gataccctgg tagtccacgc cgtaaacgat gagtgctaag tgttaggggg | 840 |
| tttccgcccc ttagtgctgc agctaacgca ttaagcactc cgcctgggga gtacggtcgc | 900 |
| aagactgaaa ctcaaaggaa ttgacggggg cccgcacaag cggtggagca tgtggtttaa | 960 |
| ttcgaagcaa cgcgaagaac cttaccaggt cttgacatcc tctgacaatc ctagagatag | 1020 |
| gacgtcccct tcgggggcag agtgacaggt ggtgcatggt tgtcgtcagc tcgtgtcgtg | 1080 |
| agatgttggg ttaagtcccg caacgagcgc aacccttgat cttagttgcc agcattcagt | 1140 |
| tgggcactct aaggtgactg ccggtgacaa accggaggaa ggtggggatg acgtcaaatc | 1200 |
| atcatgcccc ttatgacctg ggctacacac gtgctacaat ggacagaaca aagggcagcg | 1260 |
| aaaccgcgag gttaagccaa tcccacaaat ctgttctcag ttcggatcgc agtctgcaac | 1320 |
| tcgactgcgt gaagctggaa tcgctagtaa tcgcggatca gcatgccgcg gtgaatacgt | 1380 |
| tcccgggcct tgtacacacc gcccgtcaca ccacgagagt ttgtaacacc cgaagtcggt | 1440 |
| gaggtaacct tttaggagcc agccgccgaa ggtgggacag atgattgggg tgaagtcgta | 1500 |
| acaaggtagc cgtatcggaa ggtgcggctg gatcacctcc ttt | 1543 |

* Included in Table 2 are RNA nucleic acid molecules (e.g., thymidine replaced with uridines), as well as DNA or RNA nucleic acid sequences comprising a nucleic acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with the nucleic acid sequence of any SEQ ID NO listed in Table 2, or a portion thereof. Such nucleic acid molecules can have a function of the full-length nucleic acid as described further herein.

What is claimed is:

1. A method of treating or preventing macular degeneration, comprising administering to a subject in need thereof a superoxide dismutase (SOD) enzyme and probiotic Bacillus sp. spores (e.g., Bacillus coagulans, Bacillus subtilis, Bacillus indicus, Bacillus clausii, Bacillus licheniformis, Bacillus amyloliquefaciens).

2. The method of claim 1, wherein the SOD enzyme is an isolated enzyme and/or is a recombinant enzyme.

3. The method of claim 1 or 2, wherein the SOD enzyme binds manganese.

4. The method of any one of the preceding claims, wherein the SOD enzyme comprises:
(a) the amino acid sequence with at least or about 85% identity to the sequence set forth in SEQ ID NO: 1;
(b) the amino acid sequence set forth in SEQ ID NO: 1, wherein the amino acid residue Asn74 and/or Asn137 is deleted or substituted;
(c) the amino acid sequence set forth in SEQ ID NO: 1, wherein the amino acid residue Asn74 and/or Asn137 is substituted with Asp74 and/or Asp137; or
(d) the amino acid sequence set forth in SEQ ID NO: 1.

5. The method of any one of the preceding claims, wherein the SOD enzyme is coated with shellac.

6. The method of any one of the preceding claims, wherein the SOD enzyme and/or the Bacillus sp. spores are administered orally, intravenously, intraocularly, or intramuscularly.

7. The method of claim 6, wherein the SOD enzyme and/or the Bacillus sp. spores are administered orally.

8. The method of any one of the preceding claims, wherein the SOD enzyme is from a microorganism, preferably a bacterium, preferably a bacterium generally regarded as safe (GRAS) for use as food and drug, more preferably a Bacillus species bacterium.

9. The method of any one of the preceding claims, wherein the SOD enzyme is from Bacillus amyloliquefaciens GF423 strain (KCTC 13222BP) or from GF424 strain (KCTC 13227BP).

10. The method of any one of the preceding claims, wherein the probiotic Bacillus sp. spores are generally regarded as safe (GRAS) for use as food and approved drugs, preferably the spores of a Bacillus amyloliquefaciens GF423 strain or GF424 mutant strain.

11. The method of any one of the preceding claims, wherein the method
(i) decreases choroidal neovascularization (CNV);
(ii) decreases cell death in the retina;
(iii) decreases inflammation in the retina;
(iv) decreases hypoxia in the retina;
(v) decreases the expression of vascular endothelial growth factor (VEGF) in the retina; and/or
(vi) increases retinal function.

12. The method of any one of the preceding claims, wherein the macular degeneration is an age-related macular degeneration (AMD), preferably wherein the AMD is a wet AMD or a neovascular AMD.

13. The method of any one of the preceding claims, wherein the subject is a mammal, preferably wherein the mammal is a human, a dog, a cat, a mouse, or a rat.

14. The method of any one of the preceding claims, wherein the subject is a human.

15. The method of any one of the preceding claims, wherein the SOD enzyme and the probiotic Bacillus sp. spores are administered to the subject sequentially.

16. The method of any one of claims 1-14, wherein the SOD enzyme and the probiotic Bacillus sp. spores are administered to the subject simultaneously.

17. The method of claim 16, wherein the subject is administered with a composition comprising the SOD enzyme and the probiotic Bacillus sp. spores.

18. The method of any one of the preceding claims, wherein the SOD enzyme and/or the Bacillus sp. spores are in a pharmaceutical composition or a nutraceutical composition.

19. The method of any one of the preceding claims, further comprising administering to the subject at least one additional agent that treats macular degeneration.

20. The method of claim 19, wherein the at least one additional agent is ranibizumab or aflibercept.

21. A method of decreasing or inhibiting choroidal neovascularization (CNV), comprising contacting a retina with a SOD enzyme and probiotic Bacillus sp. spores (e.g., Bacillus coagulans, Bacillus subtilis, Bacillus indicus, Bacillus clausii, Bacillus licheniformis, Bacillus amyloliquefaciens).

22. The method of claim 21, wherein the SOD enzyme is an isolated enzyme and/or a recombinant enzyme.

23. The method of claim 21 or 22, wherein the SOD enzyme binds manganese.

24. The method of any one of claims 21-23, wherein the SOD enzyme comprises:
(a) the amino acid sequence with at least or about 85% identity to the sequence set forth in SEQ ID NO: 1;
(b) the amino acid sequence set forth in SEQ ID NO: 1, wherein the amino acid residue Asn74 and/or Asn137 is deleted or substituted;
(c) the amino acid sequence set forth in SEQ ID NO: 1, wherein the amino acid residue Asn74 and/or Asn137 is substituted with Asp74 and/or Asp137; or
(d) the amino acid sequence set forth in SEQ ID NO: 1.

25. The method of any one of claims 21-24, wherein the SOD enzyme is coated with shellac.

26. The method of any one of claims 21-25, wherein the SOD enzyme is from a microorganism, preferably a bacterium, preferably a bacterium generally regarded as safe (GRAS) for use as food and drug, more preferably a Bacillus species bacterium.

27. The method of any one of claims 21-26, wherein the SOD enzyme is from Bacillus amyloliquefaciens GF423 strain (KCTC 13222BP) or from GF424 strain (KCTC 13227BP).

28. The method of any one of claims 21-27, wherein the probiotic Bacillus sp. spores are generally regarded as safe (GRAS) for use as food and approved drugs, preferably the spores of a Bacillus amyloliquefaciens GF423 strain or GF424 mutant strain.

29. The method of any one of claims 21-28, wherein the method
(i) decreases cell death in the retina;
(ii) decreases inflammation in the retina;
(iii) decreases hypoxia in the retina;
(iv) decreases the expression of vascular endothelial growth factor (VEGF) in the retina; and/or
(v) increases retinal function.

30. The method of any one of claims 21-29, wherein the retina is of a subject afflicted with a macular degeneration.

31. The method of claim 30, wherein the macular degeneration is an age-related macular degeneration (AMD), preferably wherein the AMD is a wet AMD or a neovascular AMD.

32. The method of any one of claims 21-31, wherein the retina is of a mammal, preferably wherein the mammal is a human, a dog, a cat, a mouse, or a rat.

33. The method of claim 32, wherein the mammal is a human.

34. The method of any one of claims 21-33, wherein the SOD enzyme and the probiotic Bacillus sp. spores contact the retina sequentially.

35. The method of any one of claims 21-33, wherein the SOD enzyme and the probiotic Bacillus sp. spores contact the retina simultaneously.

36. The method of claim 35, wherein the retina is contacted with a composition comprising the SOD enzyme and the probiotic Bacillus sp. spores.

37. The method of any one of claims 21-36, wherein the SOD enzyme and/orthe probiotic Bacillus sp. spores are in a pharmaceutical composition.

38. The method of any one of claims 21-37, further comprising contacting the retina with at least one additional agent that decreases or inhibits CNV.

39. The method of claim 38, wherein the at least one additional agent is ranibizumab or aflibercept.

40. A pharmaceutical composition comprising a superoxide dismutase (SOD) enzyme and probiotic Bacillus sp. spores (e.g., Bacillus coagulans, Bacillus subtilis, Bacillus indicus, Bacillus clausii, Bacillus licheniformis, Bacillus amyloliquefaciens).

41. The composition of claim 40, wherein the SOD enzyme is an isolated or purified enzyme.

42. The composition of claim 40 or 41, wherein the SOD enzyme is a recombinant enzyme.

43. The composition of any one of claims 40-42, wherein the SOD enzyme binds manganese.

44. The composition of any one of claims 40-43, wherein the SOD enzyme comprises:
(a) the amino acid sequence with at least or about 85% identity to the sequence set forth in SEQ ID NO: 1;
(b) the amino acid sequence set forth in SEQ ID NO: 1, wherein the amino acid residue Asn74 and/or Asn137 is deleted or substituted;
(c) the amino acid sequence set forth in SEQ ID NO: 1, wherein the amino acid residue Asn74 and/or Asn137 is substituted with Asp74 and/or Asp137; or
(d) the amino acid sequence set forth in SEQ ID NO: 1.

45. The composition of any one of claims 40-44, wherein the SOD enzyme is coated with shellac.

46. The composition of any one of claims 40-45, wherein the composition is an oral composition.

47. The composition of any one of claims 40-46, wherein the SOD enzyme is from a microorganism, preferably a bacterium, preferably a bacterium generally regarded as safe (GRAS) for use as food and drug, more preferably a Bacillus species bacterium.

48. The composition of any one of claims 40-47, wherein the SOD enzyme is from Bacillus amyloliquefaciens GF423 strain (KCTC 13222BP) or GF424 strain (KCTC 13227BP).

49. The composition of any one of claims 40-48, wherein the probiotic Bacillus sp. spores are generally regarded as safe (GRAS) for use as food and approved drugs, preferably the spores of a Bacillus amyloliquefaciens GF423 strain or GF424 mutant strain.

50. The composition of any one of claims 40-49, further comprising at least one additional agent that decreases or inhibits CNV.

51. The composition of claim 50, wherein the at least one additional agent is ranibizumab or aflibercept.

52. The composition of any one of claims 40-51, wherein the composition
(i) decreases choroidal neovascularization (CNV);
(ii) decreases cell death in the retina;
(iii) decreases inflammation in the retina;
(iv) decreases hypoxia in the retina;
(v) decreases the expression of vascular endothelial growth factor (VEGF) in the retina; and/or
(vi) increases retinal function.

53. A medical or nutraceutical food comprising a superoxide dismutase (SOD) enzyme, and probiotic Bacillus sp. spores (e.g., Bacillus coagulans, Bacillus subtilis, Bacillus indicus, Bacillus clausii, Bacillus licheniformis, Bacillus amyloliquefaciens).

54. The medical or nutraceutical food of claim 53, wherein the SOD enzyme is an isolated or purified enzyme.

55. The medical or nutraceutical food of claim 53 or 54, wherein the SOD enzyme is a recombinant enzyme.

56. The medical or nutraceutical food of any one of claims 53-55, wherein the SOD enzyme binds manganese.

57. The medical or nutraceutical food of any one of claims 53-56, wherein the SOD enzyme comprises:
(a) the amino acid sequence with at least or about 85% identity to the sequence set forth in SEQ ID NO: 1;
(b) the amino acid sequence set forth in SEQ ID NO: 1, wherein the amino acid residue Asn74 and/or Asn137 is deleted or substituted;
(c) the amino acid sequence set forth in SEQ ID NO: 1, wherein the amino acid residue Asn74 and/or Asn137 is substituted with Asp74 and/or Asp137; or
(d) the amino acid sequence set forth in SEQ ID NO: 1.

58. The medical or nutraceutical food of any one of claims 53-57, wherein the SOD enzyme is coated with shellac.

59. The medical or nutraceutical food of any one of claims 53-58, wherein the SOD enzyme is from a microorganism, preferably a bacterium, preferably a bacterium generally regarded as safe (GRAS) for use as food more preferably a Bacillus species bacterium.

60. The medical or nutraceutical food of any one of claims 53-59, wherein the SOD enzyme is from Bacillus amyloliquefaciens GF423 strain (KCTC 13222BP) or from GF424 strain (KCTC 13227BP).

61. The medical or nutraceutical food of any one of claims 53-60, wherein the probiotic Bacillus sp. spores are generally regarded as safe (GRAS) for use as food and approved drugs, preferably the spores of a Bacillus amyloliquefaciens GF423 strain or GF424 mutant strain.

62. The medical or nutraceutical food of any one of claims 53-61, further comprising at least one additional agent that decreases or inhibits CNV.

63. The medical or nutraceutical food of claim 62, wherein the at least one additional agent is ranibizumab or aflibercept.

64. The medical or nutraceutical food of any one of claims 53-63, wherein the composition
(i) decreases choroidal neovascularization (CNV);
(ii) decreases cell death in the retina;
(iii) decreases inflammation in the retina;
(iv) decreases hypoxia in the retina;
(v) decreases the expression of vascular endothelial growth factor (VEGF) in the retina; and/or
(vi) increases retinal function.

65. A pharmaceutical composition, comprising probiotic Bacillus sp. spores (e.g., Bacillus coagulans, Bacillus subtilis, Bacillus indicus, Bacillus clausii, Bacillus licheniformis, Bacillus amyloliquefaciens).

66. The pharmaceutical composition of claim 65, wherein the probiotic Bacillus sp. spores are generally regarded as safe (GRAS) for use as food and approved drugs.

67. The pharmaceutical composition of claim 65 or 66, wherein the probiotic Bacillus sp. spores are the spores of a Bacillus amyloliquefaciens GF423 strain or GF424 mutant strain.

68. The pharmaceutical composition of any one of claims 65-67, further comprising at least one additional agent that decreases or inhibits CNV.

69. The pharmaceutical composition of claim 68, wherein the at least one additional agent is ranibizumab or aflibercept.

70. A medical or nutraceutical food, comprising probiotic Bacillus sp. spores (e.g., Bacillus coagulans, Bacillus subtilis, Bacillus indicus, Bacillus clausii, Bacillus licheniformis, Bacillus amyloliquefaciens).

71. The medical or nutraceutical food composition of claim 70, wherein the probiotic Bacillus sp. spores are generally regarded as safe (GRAS) for use as food and approved drugs.

72. The medical or nutraceutical food of claim 70 or 71, wherein the probiotic Bacillus sp. spores are the spores of a Bacillus amyloliquefaciens GF423 strain or GF424 mutant strain.

73. The medical or nutraceutical food of any one of claims 70-72, further comprising at least one additional agent that decreases or inhibits CNV.

74. The medical or nutraceutical food of claim 73, wherein the at least one additional agent is ranibizumab or aflibercept.

75. A kit comprising the pharmaceutical composition of any one of claims 40-52 and 65-69; or the medical or nutraceutical food of any one of claims 53-64 and 70-74.
